(19) United States
(12) Patent Application Publication
(10) Pub. No.: US 2023/0270119 A1
(43) Pub. Date: Aug. 31, 2023

(54) BIOSTIMULANT AND BIOPROTECTIVE PEPTIDES AND THEIR USE IN AGRICULTURE

(71) Applicant: MATERIAS S.R.L., Napoli (IT)

(72) Inventors: Rosa RAO, Napoli (IT); Francesco PENNACCHIO, Giugliano in Campania (Napoli) (IT); Donata MOLISSO, Volla (Napoli) (IT); Mariangela COPPOLA, Pomigliano d'Arco (Napoli) (IT); Ilaria DI LELIO, Sperlonga (Latina) (IT); Simona Maria MONTI, Napoli (IT); Martina BUONANNO, Casavatore (Napoli) (IT); Emma LANGELLA, Torre Del Greco (Napoli) (IT)

(21) Appl. No.: 18/006,854

(22) PCT Filed: Jul. 29, 2021
(86) PCT No.: PCT/IB2021/056897

§ 371 Date: Jan. 25, 2023

(30) Foreign Application Priority Data
Jul. 31, 2020 (IT) 102020000018811

(51) Int. Cl. A01N 63/50; A01P 21/00; A01P 3/00; A01P 1/00

(52) U.S. Cl. CPC A01N 63/50; A01P 21/00; A01P 3/00; A01P 1/00

(57) ABSTRACT

Isolated peptides having biostimulant and bioprotective activity against abiotic and biotic stress in plants are provided. Compositions comprising the peptides having biostimulant and bioprotective activity against abiotic and biotic stress in plants are also provided. Preferably, the peptides having biostimulant and bioprotective activity against abiotic and biotic stress in plants are from Solanum lycopersicum plants and are produced in a recombinant or synthetic manner. Methods for increasing resistance to biotic and/or abiotic stress in plants by applying to the plants the peptides having biostimulant and bioprotective activity and/or the compositions containing the peptides having biostimulant and bioprotective activity are further provided.

The present invention falls within the field of agriculture, more specifically of agronomic procedures aimed at promoting the growth and productivity of plants. In particular, the present invention relates to novel peptides endowed with biostimulant and bioprotective activities against abiotic and biotic stress in plants.

In the field of agriculture, it is essential to promote the growth, health, and productivity of plants. Over the years, agronomic practices have developed different approaches in order to face and stem the damage caused to plants and crops by biotic stress, i.e., the attack by microorganisms, such as viruses, bacteria, fungi, and macroorganisms, including nematodes, insects and mites, as well as by abiotic stress, such as for example lack of water, excess heat, extreme cold, or high salinity.

Traditionally, biotic and abiotic stress in plants has been controlled by using fertilizers and pesticides and introducing physical soil modifications. However, the use of agrochemicals can have serious long-term environmental consequences if these products are used in excess or remain in the environment and cause significant damage to human health due to toxic residues in the edible parts. In addition, crop protection products can lose their effectiveness over time due to the emergence of strains resistant to their action in the pathogen populations.

In order to limit the use of dangerous strategies, in an attempt to improve crop productivity by promoting plant growth and stress resistance, many efforts have been made to develop and implement ecologically cautious and sustainable approaches.

U.S. Pat. Nos. 5,378,819, 5,883,076 and 6,022,739 describe the isolation of the plant peptide hormone Systemin as well as the involvement of said peptide in the activation of defence genes in Solanum lycopersicum (tomato) plants in response to injury induced by chewing insects or mechanical damage.

Systemin is a peptide hormone of 18 amino acids in length, located at the end of the carboxy-terminal region of a 200 amino acid precursor called Prosystemin (ProSys). Following plant wounding, the precursor Prosystemin undergoes a proteolytic action probably mediated by a phytaspase, an aspartate-specific protease of the subtiliase family, which allows the release of Systemin. This peptide is released into the apoplast where, through interaction with the membrane receptor SYR1, it activates the defence signals (Narvàez-Vasquez and Orozco-Càrdenas, (2008) “Systemins and AtPeps: Defense-related peptide signals”; In Induced plant resistance to herbivory (pp. 313-328). Springer, Dordrecht; Wang L. et al., (2018) “The systemin receptor SYR1 enhances resistance of tomato against herbivorous insects”, Nature plants, 4(3), 152-156).

Under physiological conditions, the Prosystemin gene is expressed at femtomolar levels in leaves, petals and stems of plants, but not in the roots (Pearce G. et al., (1991) “A polypeptide from tomato leaves induces wound-inducible proteinase inhibitor protein”, Science, 253(5022), 895-897; Narváez-Vásquez, J., and Ryan, C. A., (2004) “The cellular localization of prosystemin: a functional role for phloem parenchyma in systemic wound signaling”, Planta, 218(3), 360-369). Conversely, in case of mechanical damage or wounding caused by the attack of chewing insects, the Prosystemin gene increases its expression.

The role of Prosystemin/Systemin in the tomato plant defence mechanisms has been extensively documented through the study of transgenic plants overexpressing the Prosystemin coding gene or silenced for the same gene. In particular, following Prosystemin overexpression, an increase in the synthesis of protease inhibiting proteins has been detected in the intestine of insects, thus greatly reducing their digestive capacity and subsequent absorption of nutrients (McGurl B. et al., (1994) “Overexpression of the ProSystemin gene in transgenic tomato plants generates a systemic signal that constitutively induces proteinase inhibitor synthesis”, Proceedings of the National Academy of Sciences, 91(21), 9799-9802). In contrast, underexpression of the Prosystemin gene results in the almost complete suppression of the production of protease inhibitors following wounding, with consequent greater susceptibility of the plant to Manduca sexta larvae (Orozco-Cardenas et al., (1993) “Expression of an antisense prosystemin gene in tomato plants reduces resistance toward Manduca sexta larvae”, Proceedings of the National Academy of Sciences, 90(17), 8273-8276).

Recent studies confirmed that plants constitutively expressing the Prosystemin gene can defend themselves against numerous biotic stresses, by activating a wide range of defensive signals (Coppola M. et al., (2015) “Prosystemin overexpression in tomato enhances resistance to different biotic stresses by activating genes of multiple signaling pathways”, Plant molecular biology reporter, 33(5), 1270-1285). In particular, these plants are capable of releasing a mixture of volatile compounds that attract predators and parasitoids of herbivorous insects, thus increasing the indirect defenses of the plant, (Corrado G. et al., (2007), “Systemin regulates both systemic and volatile signaling in tomato plants”, Journal of chemical ecology, 33(4), 669-681), are resistant to attack by necrotrophic fungi (El Oirdi et al., (2011) “Botrytis cinerea manipulates the antagonistic effects between immune pathways to promote disease development in tomato”, Plant Cell 23, 2405-2421) and aphids, tolerate some viral infections better (Bubici G. et al., (2017) “Prosystemin overexpression induces transcriptional modifications of defense-related and receptor-like kinase genes and reduces the susceptibility to Cucumber mosaic virus and its satellite RNAs in transgenic tomato plants”, PloSone, 12(2), e0171902), and at the same time are tolerant to salt stress conditions (Orsini F. et al., (2010) “Systemin-dependent salinity tolerance in tomato: evidence of specific convergence of abiotic and biotic stress responses”, Physiologia plantarum, 138(1), 10-21).

It is also known that the portion of the Prosystemin precursor protein lacking the Systemin peptide is also capable of inducing activation of defence genes. The studies described in Corrado G. et al, (2016) “The expression of the tomato prosystemin in tobacco induces alterations irrespective of its functional domain”, Plant Cell, Tissue and Organ Culture (PCTOC), 125(3), 509-519, carried out on tobacco plants which, instead of the Prosystemin gene, contain a structurally completely different, functional ortholog, have shown that, by transforming these plants with the sequence encoding Prosystemin devoid of Systemin, the deleted precursor protein is synthesized and leads to activation of a series of defence-associated genes and greater tolerance towards the fungus Botrytis cinerea.

Despite the promising results of studies on the effects of endogenous expression/overexpression of the Prosystemin protein, agronomic approaches based on plant genetic engineering procedures, however, have the undoubted limitation of requiring complex, laborious and expensive technical procedures, with consequent limitation of their scope of application. The limitations mentioned above are also accompanied by difficult legislative and regulatory issues.

Therefore, there is a need to provide methods aimed at effectively preserving and improving plant well-being and health and increasing crop quality, while being easy to implement and having minimal impact on the environment.

This and other needs have now been met by the present invention which provides an isolated peptide as defined in appended claim 1, a biostimulant and bioprotective composition as defined in appended claim 8, and a method for increasing resistance to biotic and/or abiotic stress in a plant as defined in appended claim 13.

The appended independent and dependent claims form an integral part of the present specification.

As will be illustrated in greater detail in the following experimental part, the present inventors have surprisingly isolated from the Prosystemin polypeptide and subsequently produced peptides which, when administered exogenously to a plant, for example by spraying on the foliage or irrigation, are advantageously capable of performing a growth biostimulant activity on said plant and at the same time of triggering defence mechanisms against biotic and abiotic stress, without any direct biocidal action on pathogens.

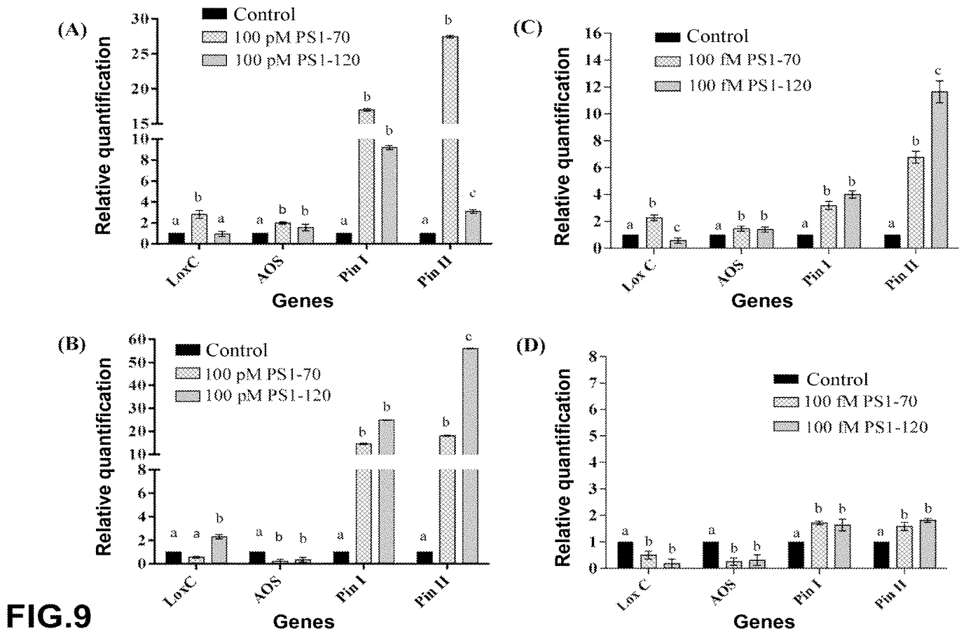

Without wishing to be bound to any theory, the present inventors believe that the resistance induced in plants following treatment with the peptides of the invention, in particular in tomato plants, against the action of noxious insects and fungi is mediated by the activation of genes involved in the regulation of the endogenous defence mechanisms of the plant (as shown in FIG. 9).

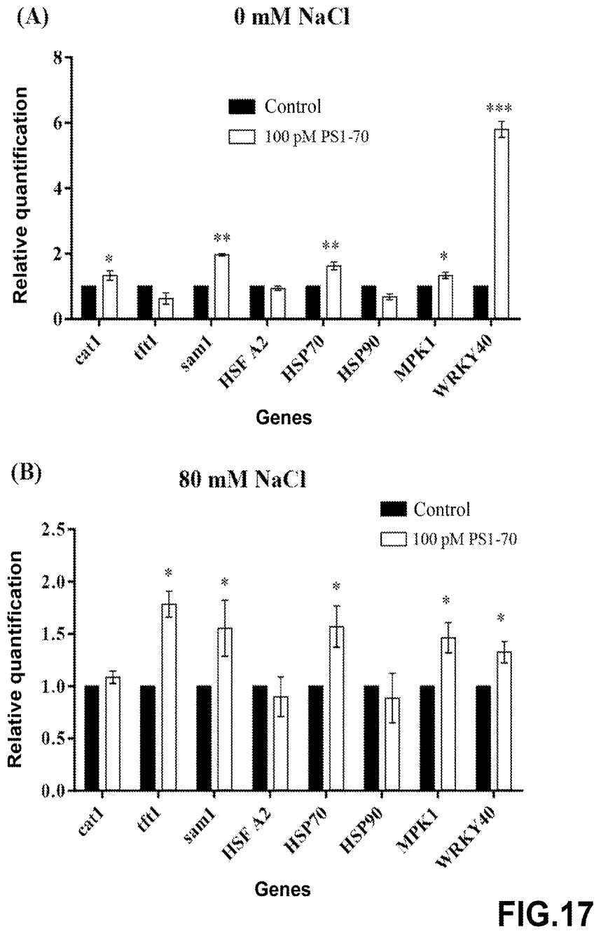

The results presented in FIGS. 17-20 also show that the peptides according to the invention are also surprisingly capable of triggering key signals of resistance to abiotic stress such as high salinity in the treated plants.

Therefore, one object of the present invention is an isolated peptide consisting of an amino acid sequence selected from the group consisting of SEQ ID NO:1, 2, 23-26 and fragments of SEQ ID NO: 2 at least 8 amino acids in length and having biostimulant and bioprotective activity against abiotic and biotic stress in plants, said peptide being optionally conjugated with a histidine tail at the amino-terminal end or at the carboxy-terminal end.

The studies carried out by the present inventors revealed that the peptides according to the invention, hereinafter referred to as PS1-70 (SEQ ID NO. 1) and PS1-120 (SEQ ID NO. 2), are contained in the amino(N-)terminal region of the Prosystemin polypeptide, more specifically in the portion of the precursor which is free of the hormone Systemin.

In the context of further studies carried out with bioinformatics approaches based on the presence of repeating amino acid motifs, the present inventors have also identified peptides consisting of the amino acid sequences DDAQEKPKVEHEEG (SEQ ID NO. 23), DKETPSQDI (SEQ ID NO. 24), DDAQEKLKVEYEEEEYEKEKIVEKETPSQDI (SEQ ID NO. 25) and DDAQEKPKVEHEEGDDKETPSQDI (SEQ ID NO. 26).

As described in Experimental example 2, due to the aberrant electrophoretic migration profiles, the identification by the inventors of the peptides object of the invention was particularly difficult and required complex investigations. The subsequent analysis of the amino acid sequences of said peptides in fact revealed the presence in said sequences of a significant component of amino acid residues known to promote structural disorder.

The term “fragment”, as used herein with reference to an amino acid sequence, refers to a continuous sequence of amino acid residues representing a portion of a longer amino acid sequence.

According to one embodiment, the isolated peptide consists of a fragment of the amino acid sequence SEQ ID NO. 2 of at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, or at least 120 amino acids in length.

In a preferred embodiment, the isolated peptide consists of a fragment of the amino acid sequence SEQ ID NO. 2 of 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 or 120 amino acids in length.

The following specific amino acid sequences are particularly preferred: EKETPSQDI (SEQ ID NO. 3), EKETISQYI (SEQ ID NO. 4), DDMQEEPKVKLHHEKG (SEQ ID NO. 5), DDTQEIPKMEHEEG (SEQ ID NO. 6), DDAQEKLKVEYEEE (SEQ ID NO. 7), DDMQEEPKVKLHHEKGGDEKEKIIEKETPSQDI (SEQ ID NO. 8) and DDTQEIPKMEHEEGGYVKEKIVEKETISQYI (SEQ ID NO. 9).

According to one embodiment, the isolated peptide object of the invention is optionally conjugated with a histidine tail at the amino-terminal (N-terminal) or carboxy-terminal (C-terminal) end.

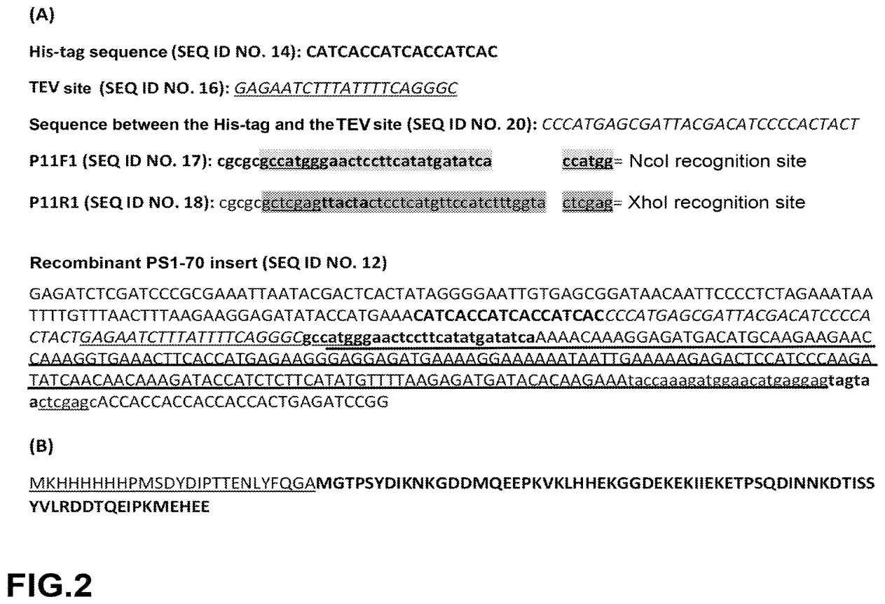

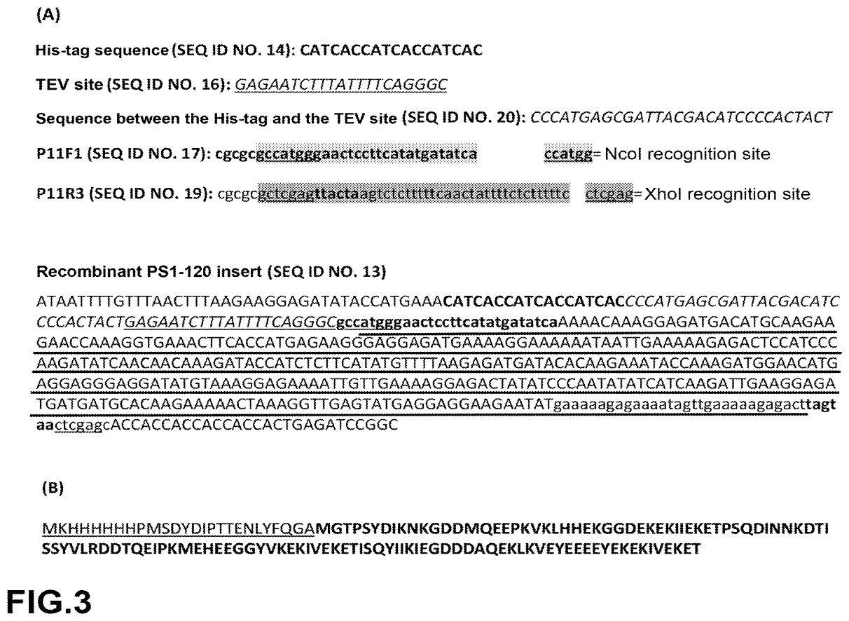

In the context of the present invention, the term “conjugated” refers to the presence of a covalent bond between the amino acid at the N-terminal end of the peptide of the invention and the amino acid at the C-terminal end of the histidine tail (as exemplified in FIGS. 2B and 3B) or, vice versa, the presence of a covalent bond between the amino acid at the C-terminal end of the peptide of the invention and the amino acid at the N-terminal end of the histidine tail, whether or not preceded by a linker.

As is known in the art, conjugation with histidine tails is routinely used in protein sciences because it simplifies protein purification procedures on matrices containing transition metal ions, and the use of anti-histidine tail antibodies is also a useful tool in localization and immunoprecipitation studies.

Methods for manufacturing peptides conjugated to a histidine tail are known and described in the state of the art, for example by expressing a recombinant protein product.

According to another embodiment, the isolated peptide object of the invention comprises an acetylation-modified amino-terminal (N-terminal) end and/or an amidation-modified carboxy-terminal (C-terminal) end. As widely described in the art, the above modifications advantageously allow the stability of a peptide and its resistance to enzymatic degradation by aminopeptidases, exopeptidases and synthetases to be increased.

A further object of the invention is an isolated nucleic acid sequence encoding an isolated peptide as defined above.

Preferably, the isolated nucleic acid sequence comprises or consists of a nucleotide sequence selected from the nucleotide sequences SEQ ID NO. 10 and SEQ ID NO. 11.

An expression vector comprising a nucleic acid sequence as defined above and optionally further comprising a promoter sequence and a polyadenylation signal sequence, as well as a host cell comprising said expression vector, are also within the scope of the present invention.

Recombinant expression vectors for use in the manufacture of peptides or proteins are known and described in the state of the art, therefore the selection and use thereof are within the skills of those of ordinary skill in the art. Such vectors may be prokaryotic or eukaryotic. Prokaryotic vectors of the PET series (Novagen) such as pET15 or pET30, and of the pGEX series (GE Healthcare) are mentioned by way of non-limiting example.

Examples of eukaryotic vectors include those of the pPIC series used in Pichia pastoris yeast cells.

Preferably, the cell system used for expressing the expression vector of the invention is selected from prokaryotic systems, for example E. coli bacterial cells.

Alternatively, the expression cell system may be a eukaryotic system, for example yeast cells such as S. cerevisiae and Pichia pastoris.

A further object of the present invention is a method for manufacturing the peptide of the invention, according to which the transformed host cell is cultured under suitable conditions and for a time sufficient for the expression of the peptide of the invention. Typically, suitable culture conditions and times depend on the cell system used and may be related, for example, to the composition of the culture medium, the pH, the relative humidity, the gaseous component of O₂ and CO₂, as well as the temperature. The selection of the most suitable culture conditions and times for use in the method of the invention is well within the knowledge and skills of those of ordinary skill in the art.

In a preferred embodiment, the method according to the invention additionally comprises the step of recovering the peptide produced from the cell culture. The recovery step can be carried out using protein purification methods which are part of the prior art, for example by means of one or more chromatographic steps, for example by affinity chromatography or size exclusion chromatography or ion exchange chromatography, or by ultrafiltration, dialysis and/or lyophilization.

Suitable alternative methods for manufacturing a peptide according to the invention include, for example, chemical synthesis procedures, or techniques for the proteolytic cleavage of the precursor protein, for example by using specific proteases or chemical agents. The selection of the most appropriate method for use within the scope of the present invention for the production of a peptide falls well within the skills of those of ordinary skill in the art.

Thanks to the advantageous features described above, the peptides according to the invention are particularly suitable for use in agronomic practices aimed at improving plant growth and crop yield, while allowing correct management of the soil and environment. In particular, the action performed by the peptides according to the invention, as it is devoid of any direct biocidal effect, advantageously does not generate harmful consequences for the useful pollinating insect populations.

The use of small molecules such as peptides represents a further advantage of the present invention since they are better suited to be designed and/or modified in order to preserve or amplify a specific activity. In addition, unlike longer proteins, such as for example the full-length Prosystemin, the small size of a peptide significantly reduces synthesis and purification costs.

Therefore, one object of the present invention is a biostimulant and bioprotective composition against abiotic and biotic stress in plants, comprising at least one peptide as defined above, or any combination thereof, and at least one adjuvant, stabilizer and/or preservative.

The at least one adjuvant, stabilizer and/or preservative in the composition is preferably conventionally used in agronomic techniques in order to allow, for example, better uniformity of distribution on plants or seeds and/or to avoid excessive foaming.

Humectants, wetting agents, and anti-foaming agents are mentioned by way of non-limiting example among the adjuvants suitable for use in the composition according to the invention.

Exemplary anti-foaming agents include mixtures of siloxanes, sorbitol, and silicon.

Exemplary humectants or wetting agents include surfactant compounds, for example sodium lauryl sulfate and betaines, mixtures of terpenes and alcohols.

Stabilizing agents in the composition of the invention include, for example, pH adjusting agents, including citric acid, acetic acid, sodium hydroxide.

Examples of preservatives suitable for use in the biostimulant and bioprotective composition of the invention include dehydroacetic acid, benzoic acid, ethylhexylglycerin, and phenoxyethanol.

The selection of the at least one adjuvant, stabilizer and/or preservative suitable for use in the composition according to the invention falls within the skills of those of ordinary skill in the art.

In one embodiment, the composition of the invention comprises at least the peptide having the amino acid sequence SEQ ID NO:1 and the peptide having the amino acid sequence SEQ ID NO:2.

In another embodiment, the composition of the invention comprises the peptide having the amino acid sequence SEQ ID NO. 3 in combination with peptides having the amino acid sequences SEQ ID NO. 5 and SEQ ID NO.8.

In yet another embodiment, the composition of the invention comprises the following combination of peptides:
a peptide having the amino acid sequence SEQ ID NO. 1;
a peptide having the amino acid sequence SEQ ID NO. 2;
a peptide having the amino acid sequence SEQ ID NO. 3;
a peptide having the amino acid sequence SEQ ID NO. 4;
a peptide having the amino acid sequence SEQ ID NO. 5;
a peptide having the amino acid sequence SEQ ID NO. 6;
a peptide having the amino acid sequence SEQ ID NO. 7;
a peptide having the amino acid sequence SEQ ID NO. 8; and
a peptide having the amino acid sequence SEQ ID NO. 9.

In a further embodiment, the composition of the invention comprises the following combination of peptides:
a peptide having the amino acid sequence SEQ ID NO. 3;
a peptide having the amino acid sequence SEQ ID NO. 4;
a peptide having the amino acid sequence SEQ ID NO. 5;
a peptide having the amino acid sequence SEQ ID NO. 6;
a peptide having the amino acid sequence SEQ ID NO. 7;
a peptide having the amino acid sequence SEQ ID NO. 8; and a peptide having the amino acid sequence SEQ ID NO. 9.

In yet a further embodiment, the composition of the invention comprises the following combination of peptides:
a peptide having the amino acid sequence SEQ ID NO. 1;
a peptide having the amino acid sequence SEQ ID NO. 2;
a peptide having the amino acid sequence SEQ ID NO. 3;
a peptide having the amino acid sequence SEQ ID NO. 4;
a peptide having the amino acid sequence SEQ ID NO. 5;
a peptide having the amino acid sequence SEQ ID NO. 6;
a peptide having the amino acid sequence SEQ ID NO. 7;
a peptide having the amino acid sequence SEQ ID NO. 8;
a peptide having the amino acid sequence SEQ ID NO. 9;
a peptide having the amino acid sequence SEQ ID NO. 23;
a peptide having the amino acid sequence SEQ ID NO. 24;
a peptide having the amino acid sequence SEQ ID NO. 25; and
a peptide having the amino acid sequence SEQ ID NO. 26.

According to the official definition formulated by the European Biostimulant Industry Council (EBIC), the term “biostimulant” refers to a substance and/or a microorganism whose function, when applied to plants or to the rhizosphere, is to stimulate the natural process to improve/promote nutrient absorption, nutrient efficiency, crop quality, and to tolerate abiotic stress.

Within the scope of the present invention, the term “bioprotector” refers to a substance and/or a microorganism which, following administration to plants or to the rhizosphere, induces the activation of the plants' natural defenses against biotic and abiotic stress.

The term “plant”, as used herein, refers to a living multicellular plant organism.

Preferably, the plant belongs to a family selected from the group consisting of Solanacee, such as for example Solanum lycopersicum and Solanum melongena, Vitacee, such as for example Vitis vinifera, Rosaceae, such as for example Malus domestica, Oleaceae, such as for example Olea europaea, and combinations thereof.

With reference to the biotic stress, herbivorous insects, phytopathogenic fungi, phytopathogenic bacteria and viruses are mentioned by way of non-limiting example.

In this context, herbivorous insects are preferably selected from the group consisting of lepidoptera, such as for example Spodoptera littoralis and Tuta absoluta, phytomites such as aphids, for example Macrosiphum euphorbiae, homoptera such as for example Bemisia tabaci and Trialeurodes vaporariorum, and combinations thereof.

Phytopathogenic fungi are preferably selected from the group consisting of Botrytis cinerea, Alternaria alternata, Alternaria solani, and combinations thereof.

Phytopathogenic bacteria are preferably Pseudomonas syringae bacteria.

Viruses are preferably selected from Tomato spotted wilt virus and Cucumber Mosaic Virus.

Low temperatures, which cause, for example, freezing, high temperatures, drought, high light intensity, low light intensity, excess salinity, excess water, and combinations thereof are mentioned for example, although not exclusively, among the abiotic stresses.

Preferably, the composition according to the invention also comprises a buffering agent. A phosphate buffer, even more preferably a phosphate-buffered saline, is particularly preferred among the buffering agents suitable for use in the biostimulant and bioprotective composition of the invention. However, it is to be understood that other buffering agents may be used in the present invention, the selection of which falls within the skills of those of ordinary skill in the art.

Preferably, the at least one peptide is present in the biostimulant and bioprotective composition according to the invention in a concentration ranging from 0.02 picomolar (pM) to 100 pM, more preferably from 0.02 pM to 0.08 pM, or from 0.085 pM to 0.1 pM, or from 0.095 to 0.25 pM, or from 1 pM to 100 pM.

According to a preferred embodiment of the invention, the biostimulant and bioprotective composition also includes a microorganism selected from the group consisting of mycorrhizal fungi, saprophytic fungi, plant growth promoting bacteria, Bacillus thuringiensis spores, and any combination thereof.

As is known in the art, underground mycorrhizal fungi establish symbiotic associations with the roots of many crops, with mutual benefits for the organisms involved. More specifically, mycorrhizal fungi are able to metabolize the mineral elements present in the soil even if they are fixed to the absorbing power of the soil, while the plant provides the symbiotic fungi with the sugars produced by photosynthesis.

Within the scope of the present invention, the mycorrhizal fungus is preferably selected from the group consisting of Gigaspora fasciculatus, Glomus constrictum, Glomus tortuosum, Glomus geosporum, Gigaspora margarita, Acaulospora scrobicurata, and any combination thereof.

Within the scope of the invention, the saprophytic fungus preferably belongs to the genus Trichoderma.

Saprophytic fungi are known for their beneficial activity of degradation of dead plants and animals in the soil.

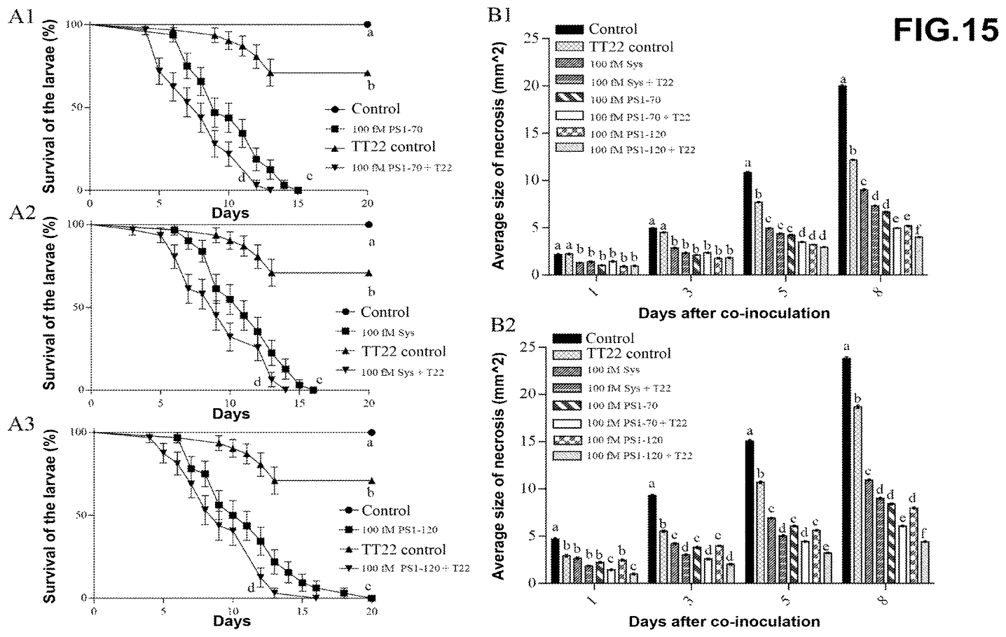

According to the invention, a biostimulant and bioprotective composition comprising at least one peptide as defined above in combination with a saprophytic fungus belonging to the genus Trichoderma is highly preferred since said combination, as shown in FIG. 15, has a marked synergistic effect against plant pathogens.

Within the scope of the invention, the bacterium which promotes plant growth is preferably selected from Burkholderia cepacia and Pseudomonas fluorescens.

Bacillus thuringiensis is a sporogenous bacterium naturally found in the soil, known to produce, under unfavourable conditions, a spore and a parasporal body, commonly called crystal, containing endotoxins with insecticidal action. These, after ingestion by sensitive insects, are released from the parasporal bodies and cause the lysis of intestinal epithelial cells with consequent paralysis and death of the insect.

More preferably, the spores in the composition according to the invention are from the bacterium Bacillus thuringiensis subspecies aizawai.

According to the present invention, the biostimulant and bioprotective composition may be in the form of a lyophilizate. In this embodiment, the composition according to the invention is stable at room temperature for at least 3 months.

In another embodiment, the composition of the present invention may be in the form of a water-based liquid composition or a phosphate-buffered saline. In this form, the composition according to the invention can be used as it is or diluted prior to use.

A method for increasing resistance to biotic and/or abiotic stress in a plant, comprising the step of applying a biostimulant and bioprotective composition as defined above to the plant, parts of the plant, plant propagation material, and/or plant growth site, is also within the scope of the invention.

According to the method of the invention, the biostimulant and bioprotective composition can be applied to a variety of plants in various forms or parts of a plant, such as for example leaves, gems, branches, stems, bark, flowers, flower buds, fruits, roots, seeds, bulbs, tubers and/or sprouts.

The term “propagation material” as used herein refers to any plant material from which a plant or part of a plant can be derived. Seeds, seedlings, cuttings, scions, rootstocks, explants, bulbs, tubers, and combinations thereof, are mentioned by way of non-limiting example.

Additionally, or alternatively, the composition according to the invention can be applied to the plant growth site.

In one embodiment, the plant is grown in the soil and the application of the composition according to the invention can take place, for example, over the entire cultivation surface, in one or more furrows and/or around them, in the sowing holes, in the area below the stem or trunk, and/or in the area between the roots.

In another embodiment, the plant is grown out of the soil or soilless. The hydroponic cultivation technique is mentioned by way of non-limiting example among the out-of-the-soil or soilless cultivation methods, in which soil is replaced by an inert substrate, such as for example expanded clay, coconut fibre, rock wool or zeolite, and the plant assimilates nutrients thanks to a solution composed of water and inorganic elements such as for example Mg(NO₃)₂·6H₂O, Ca(NO₃)₂·4H₂O, KNO₃, K₂SO₄, KH₂PO₄, which have the purpose of providing all the substances required for the normal mineral nutrition of the plant organism. A great advantage of the hydroponic cultivation practice is that this cultivation technology allows a constant and controlled production throughout the year, both from a qualitative point of view and from a hygienic and sanitary point of view due to the absence of pesticides, herbicides, and plant protection products.

The biostimulant and bioprotective composition of the invention can be applied to the plant, parts of the plant, plant propagation material, and/or plant growth site by conventional methods, for example by spraying, atomizing, sprinkling, spreading, or irrigating (by hand, with a tractor, with a plane, and the like).

According to a preferred embodiment, the composition of the present invention is applied by spraying or atomizing on the plant or parts of the plant, preferably on the leaves.

According to another preferred embodiment, the composition of the present invention is applied by irrigation, i.e., directly into the soil, for example in the form of an irrigation liquid or by injection into the soil.

In the case of a hydroponic cultivation, the method according to the invention provides that the biostimulant and bioprotective composition is administered to the plants in the nutrient solution.

According to one embodiment of the invention, the method comprises applying the composition at least twice, preferably 4 times, more preferably 5 times.

In this embodiment, the time interval between one application on the plant, for example a first, second, third, fourth or fifth application, and the subsequent application, can range from about 3 weeks to about 4 weeks.

A further object of the present invention is the use of an isolated peptide as previously defined, or of a biostimulant and bioprotective composition as previously defined, to increase the resistance to biotic and/or abiotic stress in a plant.

The experimental section that follows is provided for illustration purposes only and does not limit the scope of the invention as defined in the appended claims. In the experimental section, reference is made to the accompanying drawings, wherein:

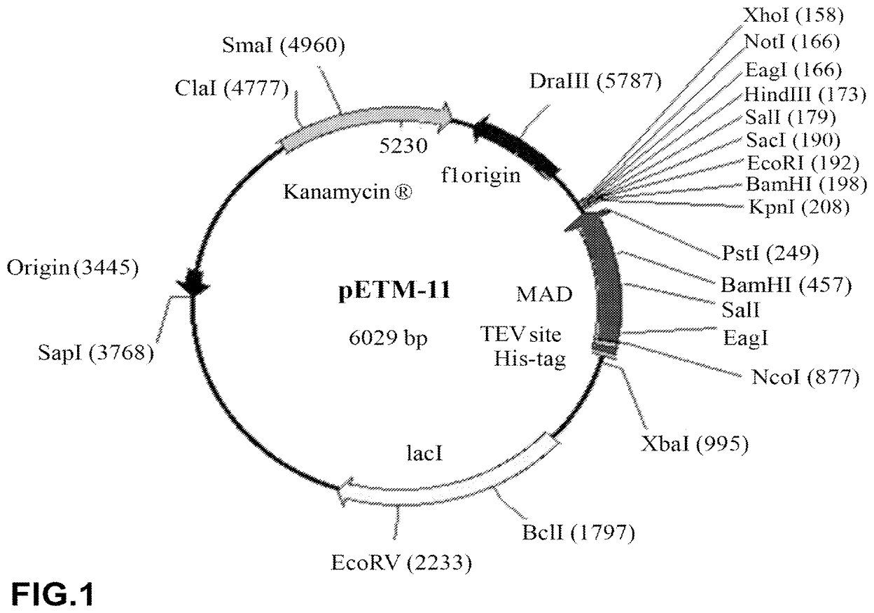

FIG. 1 shows a schematic representation of the cloning vector pETM11 used by the present inventors for the recombinant production of the peptides of the invention.

FIG. 2 shows a schematic representation of the insert containing the nucleotide sequence encoding the peptide PS1-70 after cloning into the vector pETM11. (A) Sequence of the recombinant insert (SEQ ID NO. 12) obtained by Sanger sequencing. The nucleotide sequence encoding the peptide PS1-70 (SEQ ID NO. 10), correctly inserted in the cloning vector, is underlined in black; the sequence encoding for the histidine tail (His-tag) (SEQ ID NO. 14) is highlighted in bold uppercase letters; the Tobacco Etch Virus (TEV) protease recognition site (SEQ ID NO. 16), placed downstream of the histidine tail to allow the removal of the latter, is highlighted in uppercase italics underlined in grey. The sequences of the forward primer P11F1 (SEQ ID NO. 17) and of the reverse primer P11R1 (SEQ ID NO. 18) used for amplifying and cloning the nucleotide sequence encoding the peptide PS1-70 are highlighted in light grey and dark grey, respectively. B) Amino acid sequence of the peptide PS1-70 (in bold, SEQ ID NO. 1), conjugated at the N-terminal position to the C-terminal end of a histidine tail (underlined sequence, SEQ ID NO. 15).

FIG. 3 shows a schematic representation of the insert containing the nucleotide sequence encoding the peptide PS1-120 after cloning into the vector pETM11. (A) Sequence of the recombinant insert obtained by Sanger sequencing (SEQ ID NO. 13). The nucleotide sequence encoding the peptide PS1-120 (SEQ ID NO. 11), correctly inserted in the cloning vector, is underlined in black; the sequence encoding for the histidine tail (His-tag) (SEQ ID NO. 14) is highlighted in bold; the Tobacco Etch Virus (TEV) protease recognition site (SEQ ID NO. 16), placed downstream of the histidine tail to allow the removal of the latter, is highlighted in uppercase italics underlined in grey. The sequences of the forward primer P11F1 (SEQ ID NO. 17) and of the reverse primer P11R3 (SEQ ID NO. 19) used for amplifying and cloning the nucleotide sequence encoding the peptide PS1-120 are highlighted in light grey and dark grey, respectively. (B) Amino acid sequence of the peptide PS1-120 (in bold, SEQ ID NO. 2), conjugated at the N-terminal position to the C-terminal end of a histidine tail (underlined sequence, SEQ ID NO. 15).

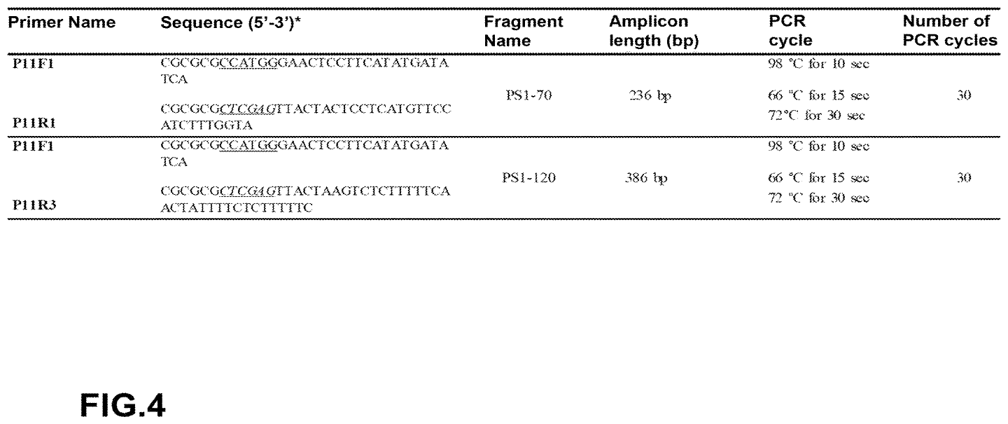

FIG. 4 is a table showing the nucleotide sequences and characteristics of the primers used for amplifying and cloning the nucleotide sequences encoding the peptides PS1-70 and PS1-120, respectively.

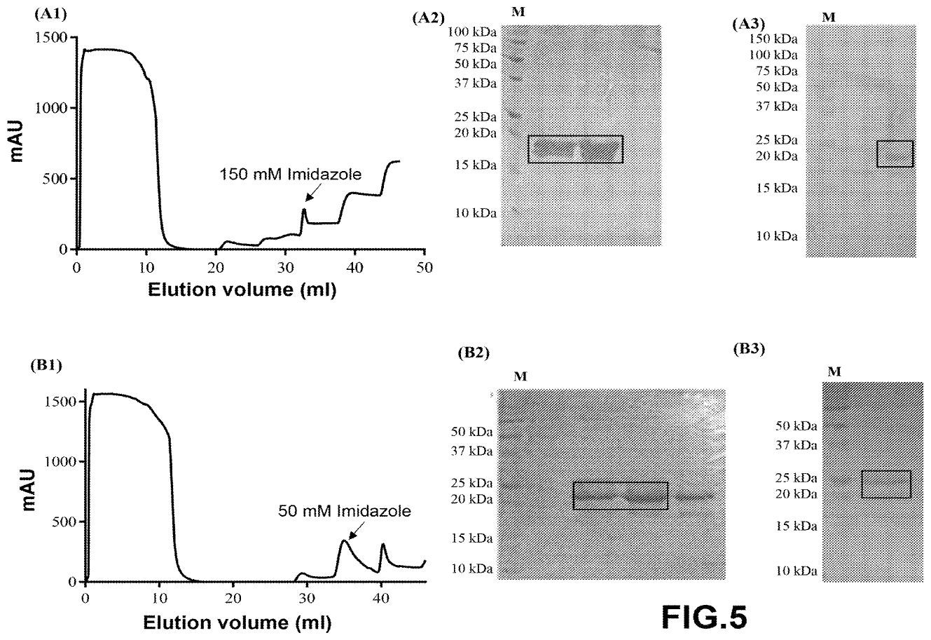

FIG. 5 shows the results of the affinity chromatography (IMAC), of the analysis of the eluted fractions by electrophoresis with 15% SDS-PAGE and of the Western blot analysis performed on the purified PS1-70 and PS1-120 peptides. (A1 and B1) Chromatographic profiles of the first step of purification of the peptides PS1-70 and PS1-120, which elute with 150 mM and 50 mM imidazole, respectively. (A2 and B2) Polyacrylamide gel analysis of the eluted fractions; M: molecular weight marker; black rectangle: eluted fractions containing the peptides PS1-70 and PS1-120. (A3 and B3) Identification of the peptides PS1-70 and PS1-120 by Western Blot analysis; M: molecular weight marker; black rectangle: PS1-70 and PS1-120 peptides.

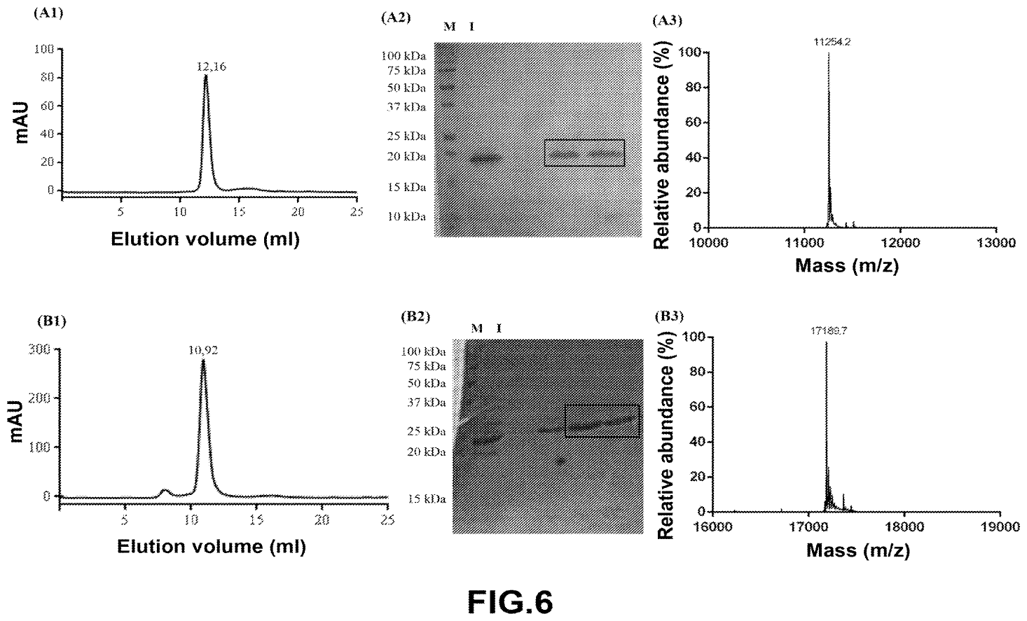

FIG. 6 shows the results of the size exclusion chromatography (SEC) and of the analysis of the eluted fractions by electrophoresis with 15% SDS-PAGE. (A1 and B1) Chromatographic profiles of the second step of purification of the peptides PS1-70 and PS1-120 which have their elution peak at an elution volume of 12.16 ml and 10.92 ml, respectively. (A2 and B2) Polyacrylamide gel analysis of the eluted fractions; M: molecular weight marker; black rectangle: eluted fractions containing the peptides PS1-70 and PS1-120. (A3 and B3) Deconvoluted mass of the peptides PS1-70 and PS1-120.

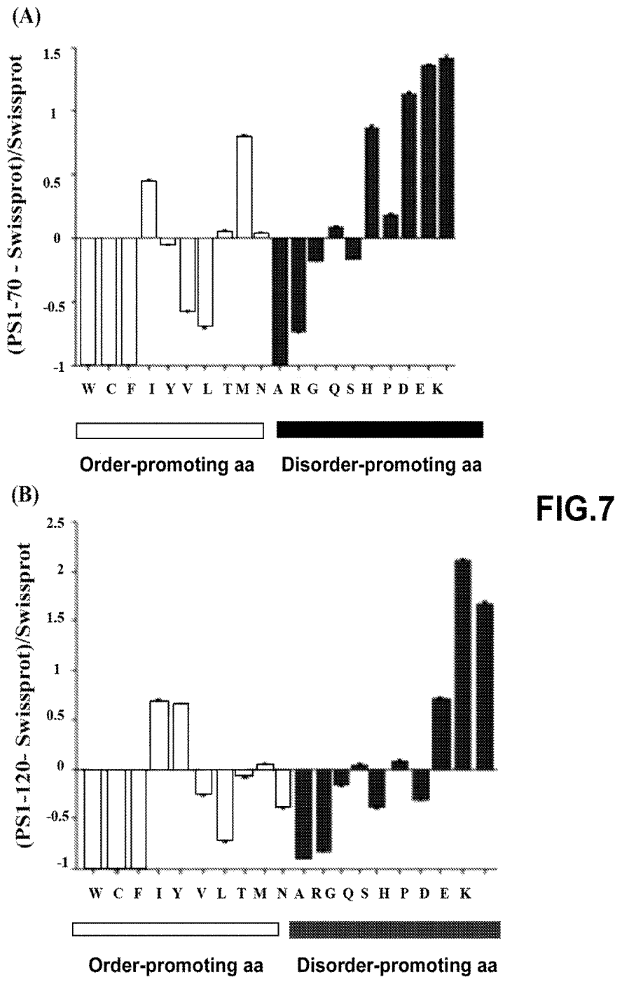

FIG. 7 shows the profiles of the amino acid composition of the peptides PS1-70 (A) and PS1-120 (B). The graphs of the figure indicate that the amino acid sequences of said peptides both contain a significant representation of amino acids promoting structural disorder (dark grey) compared to amino acids promoting an ordered secondary structure (light grey).

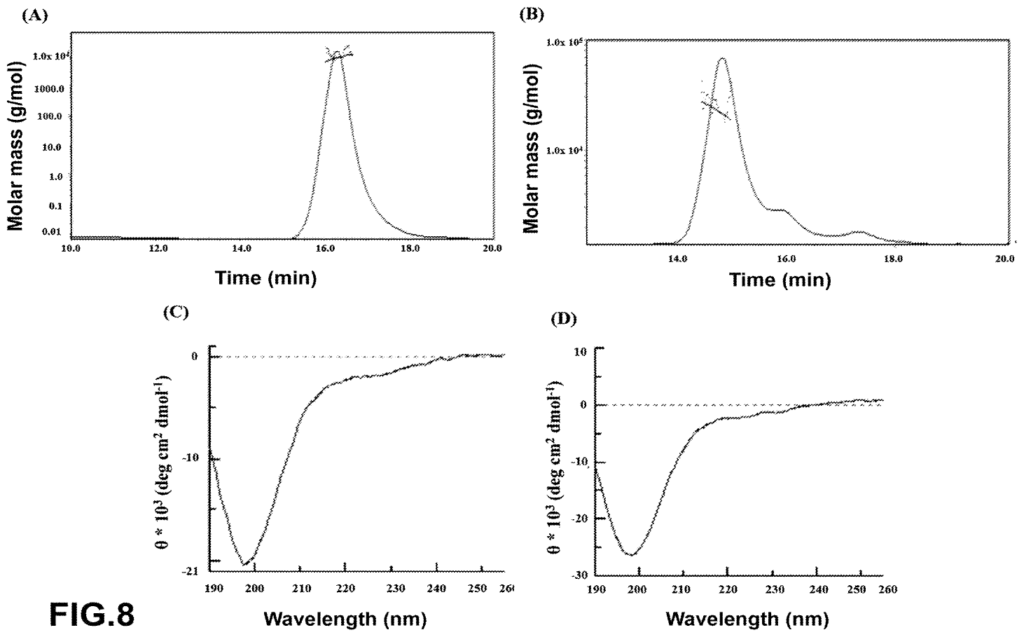

In FIG. 8, graphs A and B illustrate the results of the Light Scattering experiments carried out by SEC-MALS-QELS on the peptides PS1-70 (A) and PS1-120 (B) at pH 8.0 as described in Examples 1 and 2. The peaks of the curves are representative of monomeric proteins in solution. FIG. 8 (C,D) shows the dichroic spectra of the purified PS1-70 (C) and PS1-120 (D) peptides recorded at the temperature of 20° C. using the peptides PS1-70 and PS1-120 at concentrations of 4.4 μM and 3.5 μM, respectively, in 10 mM phosphate buffer. The abscissa axis shows the wavelength (nm), the ordinate axis shows the mean residue molar ellipticity value.

FIG. 9 shows the relative quantification of induced gene expression in tomato plants 6 hours (A,C) and 24 hours (B,D) after foliar application of peptides PS1-70 and PS1-120 at 100 pM and 100 fM concentrations. The analysis was carried out on the Lox C, AOS, Pin I and Pin II genes. The letters a, b, c indicate the statistical significance of the data (ANOVA), each letter representing a statistical group.

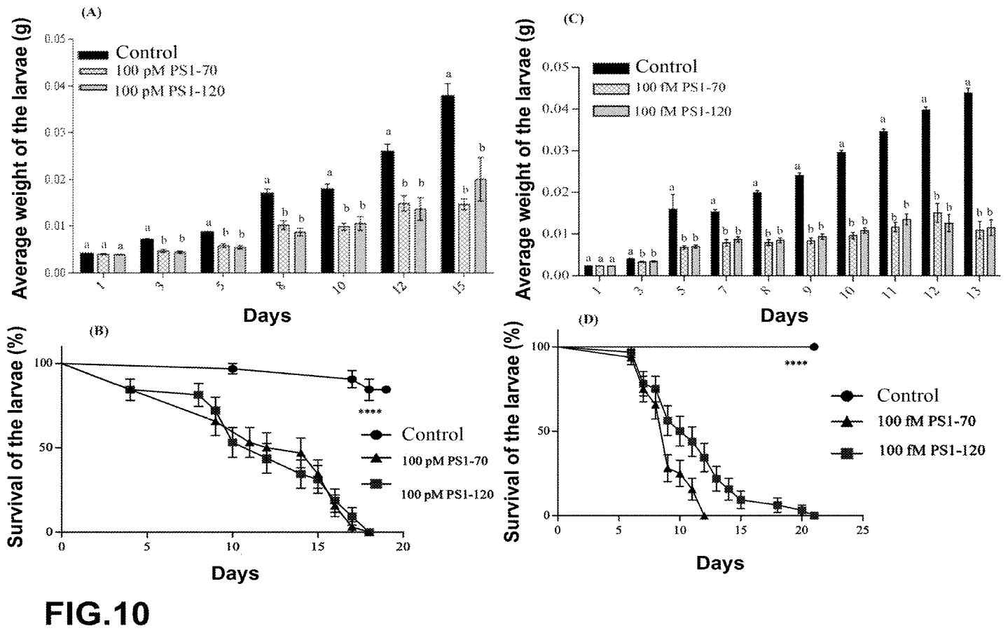

FIG. 10 illustrates the effects of the treatment of tomato plant leaves with the peptides PS1-70 and PS1-120 on S. littoralis lepidopteran larvae. The histograms (A,C) show the change in average weight, expressed in grams, of larvae fed with leaves treated with the peptides PS1-70 and PS1-120 at 100 pM and 100 fM, respectively, and the related controls, measured on several subsequent days. The letters a, b indicate the statistical significance of the data (ANOVA), each letter representing a statistical group. The graphs (B,D) show the mortality rate recorded each day for larvae fed with tomato plant leaves treated with the peptides of the invention and the related control (Log-Rank test; ****p<0.0001).

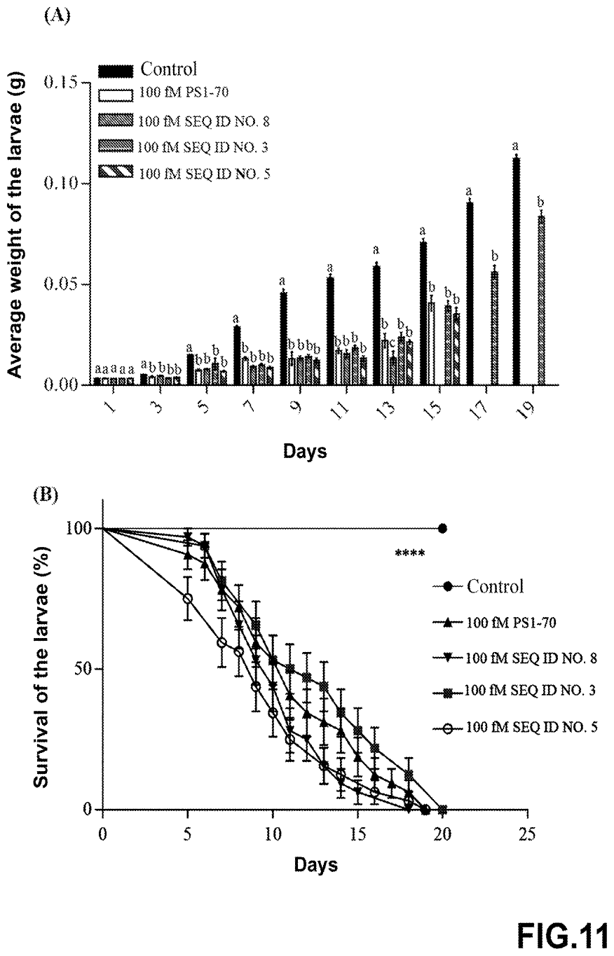

FIG. 11 illustrates the effects of the treatment of tomato plant leaves with the peptides PS1-70 and SEQ ID NO. 3, 5 and 8 on S. littoralis lepidopteran larvae. The histogram (A) shows the change in average weight, expressed in grams, of larvae fed with leaves treated with the above mentioned peptides and the related control, measured on days 1, 3, 5, 7, 9, 11, 13, 15, 17, and 19. The letters a, b indicate the statistical significance of the data (ANOVA), each letter representing a statistical group. The graph (B) shows the daily mortality rate recorded for larvae fed with tomato plant leaves treated with the peptides of the invention and the related control (Log-Rank test; ****p<0.0001).

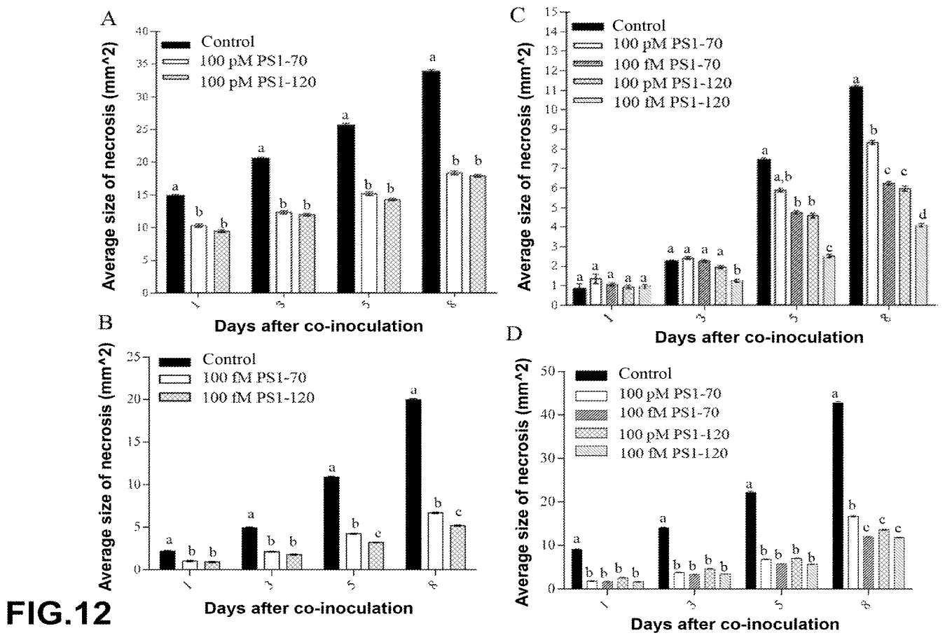

FIG. 12 shows the decrease in the necrosis areas generated by the necrotrophic B. cinerea fungus on tomato plant leaves (A,B), eggplant leaves (C) and vine plant leaves (D) after treatment with the PS1-70 and PS1-120 peptides, compared to untreated controls. The mean necrosis areas were measured 1, 3, 5 and 8 days after inoculation of the pathogen. The letters a, b, c, d indicate the statistical significance of the data (ANOVA), each letter representing a statistical group.

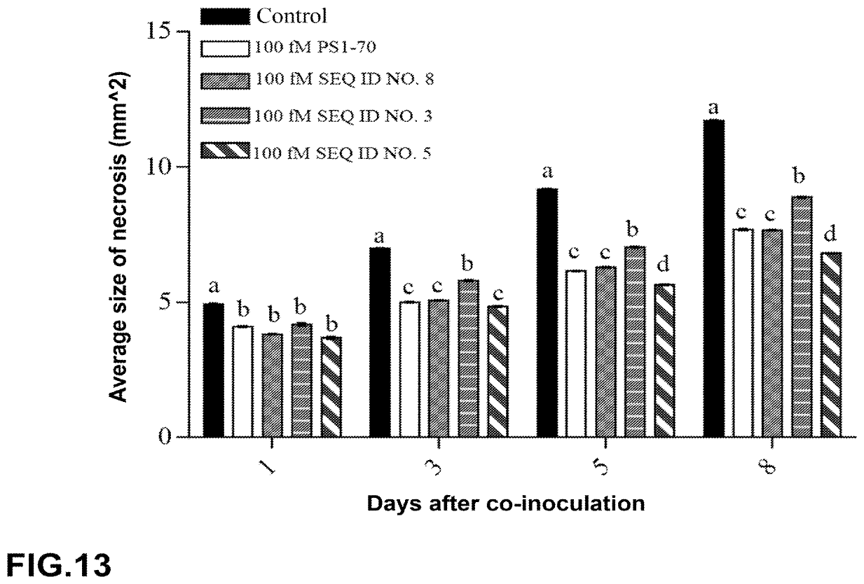

FIG. 13 shows the decrease in the necrotic areas generated by the necrotrophic B. cinerea fungus on tomato plant leaves after treatment with PS1-70, SEQ ID NO. 3, 5 and 8 compared to the untreated control. The mean necrosis areas were measured 1, 3, 5 and 8 days after inoculation of the pathogen. The letters a, b, c, d indicate the statistical significance of the data (ANOVA), each letter representing a statistical group.

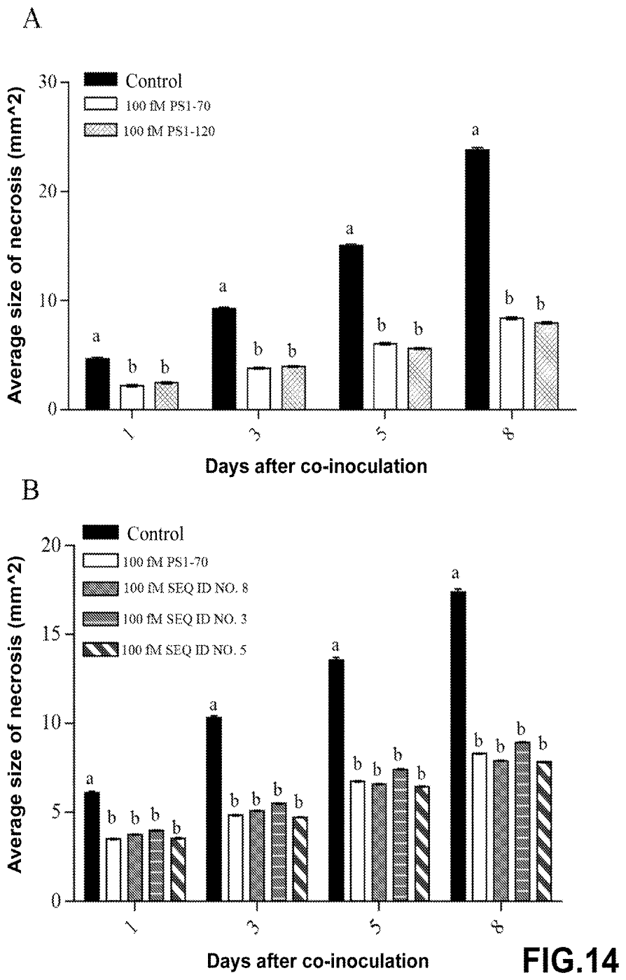

FIG. 14 shows the decrease in the necrosis areas generated by the necrotrophic A. alternata fungus on tomato plant leaves after treatment with the peptides PS1-70 and PS1-120 (A), and with PS1-70, SEQ ID NO. 3, 5 and 8 (B), compared to the untreated control. The mean necrosis areas were measured 1, 3, 5 and 8 days after inoculation of the pathogen. The letters a, b indicate the statistical significance of the data (ANOVA), each letter representing a statistical group.

FIG. 15 shows the effects of the treatment with the peptides PS1-70, PS1-120 and Systemin (Sys) in combination with Trichoderma harzianum T22 strain spores on 4-week plants born from seeds co-infected with Trichoderma T22 spores, on the survival of S. littoralis lepidopteran larvae (A1, A2 and A3), and on leaf colonization by necrotrophic B. cinerea (B1) and A. alternata (B2) fungi. Larval survival was measured daily (Log-Rank test; ****p<0.0001), each letter representing a statistical group. The mean necrosis areas were measured 1, 3, 5 and 8 days after inoculation of the pathogens. The letters a, b, c, d, e, f indicate the statistical significance of the data (ANOVA), each letter representing a statistical group.

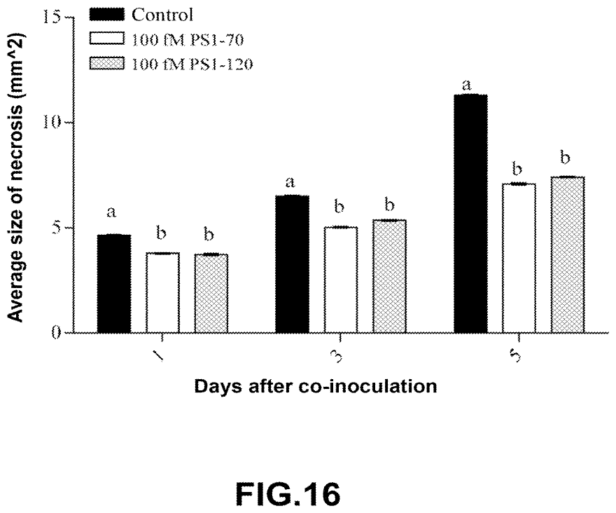

FIG. 16 shows the decrease in the necrosis areas generated by the necrotrophic B. cinerea fungus on four-week-old tomato plant leaves born from seeds treated with a 100 fM suspension of the peptides PS1-70 and PS1-120, and the respective control. The mean necrosis areas were measured 1, 3 and 5 days after inoculation of the pathogen. The letters a, b indicate the statistical significance of the data (ANOVA), each letter representing a statistical group.

FIG. 17 shows the relative quantification of induced gene expression in tomato plants irrigated with the PS1-70 peptide at a concentration of 100 pM, in the absence of salt (A) (0 mM NaCl) and in the presence of salt (B) (80 mM NaCl). The analysis was carried out on the cat1, tft1, Sam, HSFA2, HSP70, HSP90, MPK1 and WRKY40 genes. The asterisks indicate the statistical significance of the data by Student's t-test (*p<0.05; **p<0.01; ***p<0.001).

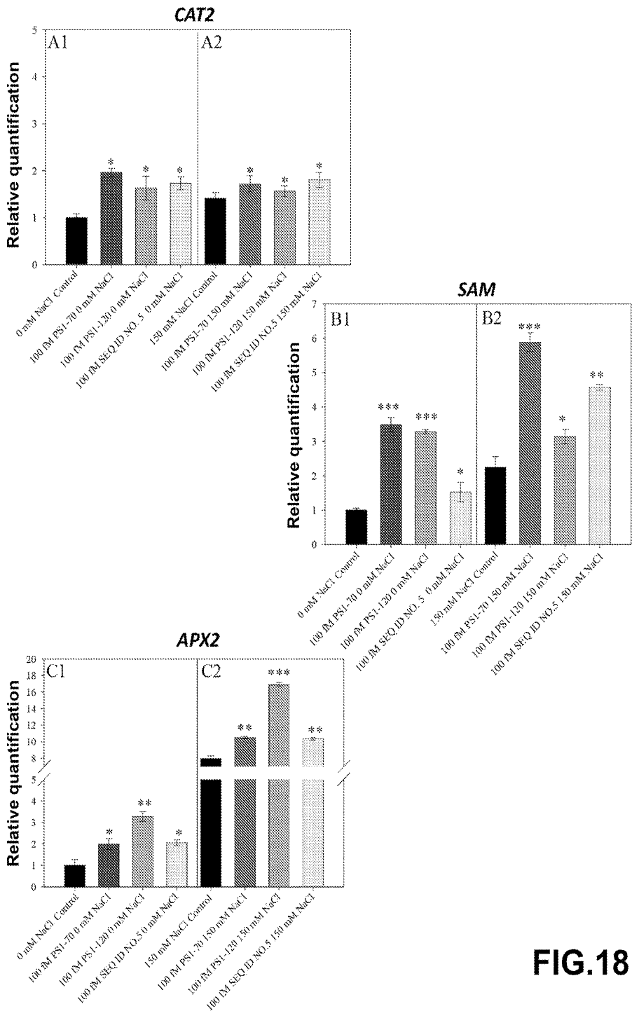

FIG. 18 shows the relative quantification of induced gene expression in tomato plants irrigated with the peptides PS1-70, PS1-120 and SEQ ID NO.5 (100 fM) in the absence of salt (0 mM NaCl), in the presence of salt (150 mM NaCl), and related controls. The analysis was carried out on the CAT2(A), SAM (B) and APX2 (C) genes. The asterisks indicate the statistical significance of the data by Student's t-test (*p<0.05; **p<0.01; ***p<0.001).

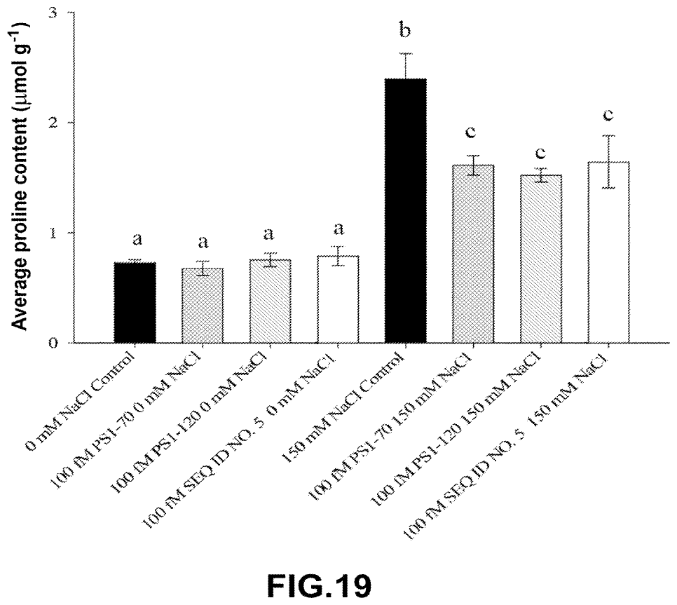

FIG. 19 shows the average proline content in plant leaves irrigated with the peptides PS1-70, PS1-120 and SEQ ID NO. 5 (100 fM) in the absence of salt (0 mM NaCl), in the presence of salt (150 mM NaCl), and related controls. The letters a, b, c indicate the statistical significance of the data (ANOVA), each letter representing a statistical group.

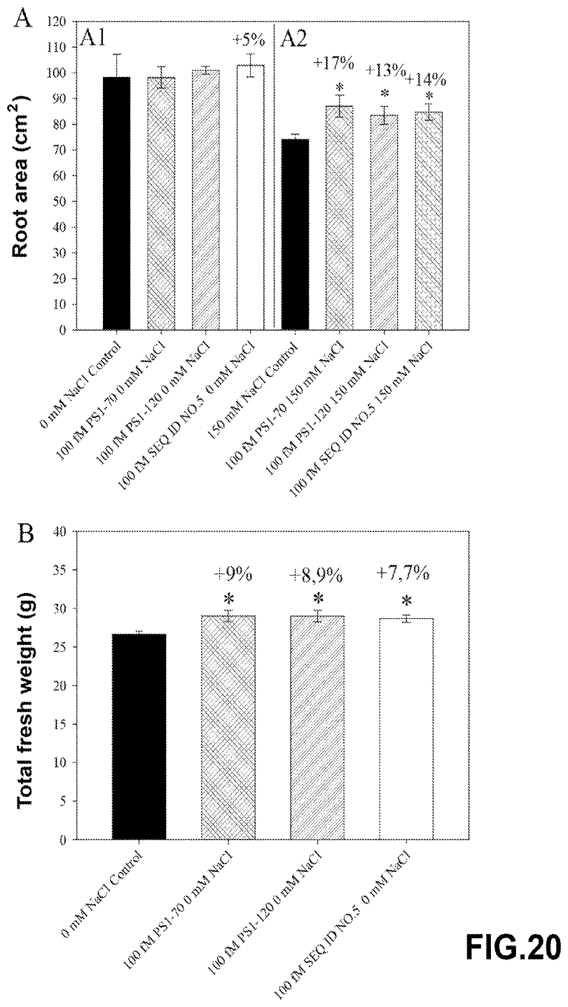

FIG. 20 illustrates the effect of irrigation treatment with the peptides PS1-70, PS1-120 and SEQ ID NO.5 at a concentration of 100 fM on the biometric parameters of the tomato plant. The histogram (A) shows the root area, expressed in square centimetres, of the plants treated with the peptides object of the invention, and the related controls, in the absence of salt (0 mM NaCl) and in the presence of salt (150 mM NaCl), and related controls. The histogram (B) shows the change in fresh weight, expressed in grams, of the aerial part in plants treated with the peptides object of the invention, and the related controls, in the absence of salt (0 mM NaCl). The asterisks indicate the statistical significance of the data by Student's t-test (*p<0.05).

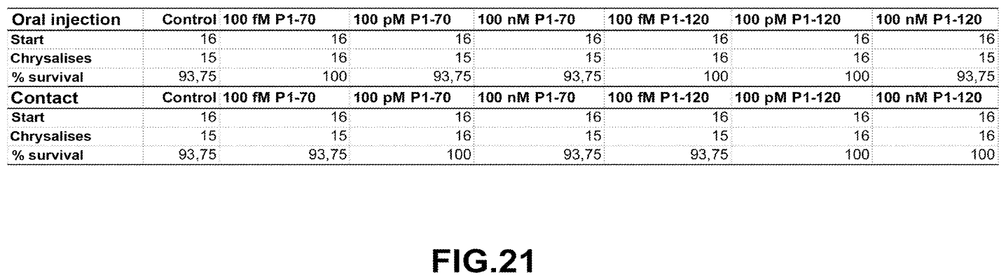

FIG. 21 shows a table with the results of the assessment of the direct toxic effect of the peptides of the invention assayed at increasing concentrations on S. littoralis larvae, as shown in Example 4. The survival rate was recorded up to the chrysalis stage for the larvae in which the peptides PS1-70 and PS1-120 were injected or applied to the epidermis.

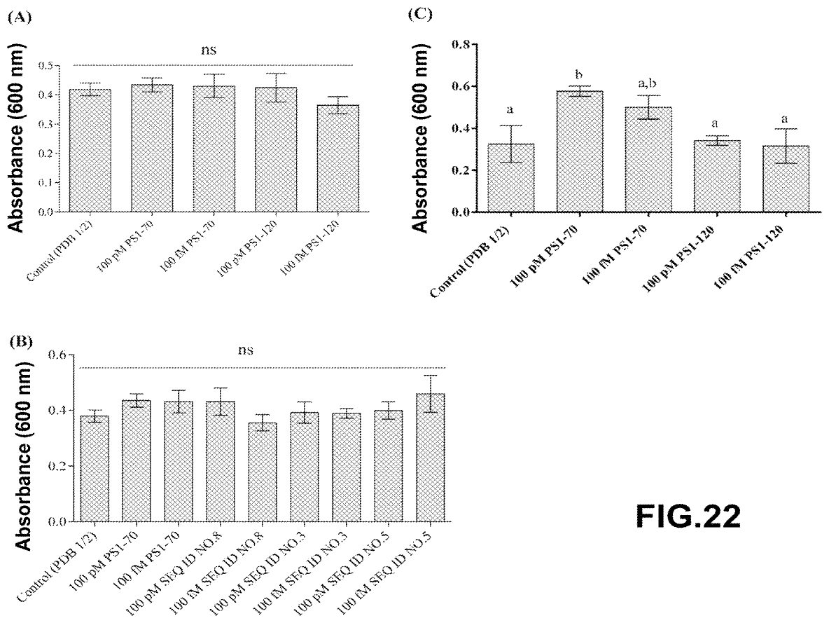

FIG. 22 shows the assessment of the direct toxic effect of the peptides of the invention assayed at increasing concentrations when added to the growth medium of two different fungi: B. cinerea, (A) PS1-70 and PS1-120 and (B) PS1-70, SEQ ID NO. 3, 5 and 8, and Trichoderma T22, (C) PS1-70 and PS1-120. The growth of the fungus was measured 24 hours after the addition of the peptides of the invention as the turbidity level of the medium (absorbance at 600 nm). The letters a, b indicate the statistical significance of the data (ANOVA), each letter representing a statistical group.

### Example 1: Production of the Peptides According to the Invention

Cloning, Expression and Purification

In order to isolate the nucleotide sequences encoding the peptides of the invention, PCR reactions were set up using the amplification primer pairs whose sequences are shown in the table of FIG. 4 and using the cDNA encoding for full-length Prosystemin as a template.

Amplicons were digested using NcoI and XhoI restriction enzymes, and subsequently cloned into the pETM11 vector previously digested with the same enzymes. pETM11 (courtesy of EMBL, Heidelberg) is a procaryotic expression vector, which is capable of adding a tail of six histidines (His-tag) at the amino(N-)terminal portion of the cloned protein and has a TEV (Tobacco Etch Virus) protease recognition site downstream of the His-tag sequence to allow removal of the latter (FIG. 1).

The integrity of the cloned fragments and the absence of possible mutations occurred during the amplification reaction were confirmed by sequencing the obtained constructs (FIGS. 2A and 3A). After an initial screening, large-scale expression of the peptides of the invention PS1-70 and PS1-120 was carried out in Escherichia coli BL21(DE3) strain for 16 hours at 22° C. in the presence of 2 mM IPTG in LB and 2-YT culture media. FIGS. 2B and 3B, respectively, show the amino acid sequences of the obtained peptides PS1-70 (SEQ ID NO. 1) and PS1-120 (SEQ ID NO. 2), highlighted in bold, each conjugated at the N-terminal end to the C-terminal end of a histidine tail, highlighted with underscores.

Purification of peptides after expression was performed at room temperature on FPLC-ÄKTA (GE Healthcare) by affinity chromatography (IMAC) (FIG. 5) and size exclusion chromatography (SEC) (FIG. 6) obtaining yields of 2 mg/L cell culture.

Peptide Synthesis

The peptides of the invention having the sequences SEQ ID NO: 3, 5 and 8 were produced by solid phase chemical synthesis using standard protocols (Chandrudu S. et al, “Chemical methods for peptide and protein production”; Molecules. 2013 Apr. 12; 18(4):4373-88). This procedure involved the use of a resin, which made it possible to obtain peptides modified by amidation at the carboxy-terminal end. At the end of the synthesis, the amino-terminal end of the peptides was also modified by acetylation. Purifications were carried out by reverse phase HPLC.

### Example 2: Analysis of the Structure of the Peptides According to the Invention

During the procedure for the identification of the peptides of the invention, the present inventors encountered considerable difficulties due to the peculiar characteristics of the PS1-70 and PS1-120 peptides, primarily the significant presence in their primary sequence of amino acid residues promoting structural disorder (FIG. 7). Both peptides PS1-70 and PS1-120 exhibited aberrant migration when subjected to SDS-PAGE electrophoresis, migrating with an apparent molecular weight of 20-25 kDa relative to their actual weight (MW PS1-70=11 kDa; MW PS1-120=17 kDa) and again, mass spectrometry confirmed the exact molecular weight of the two recombinant peptides (FIGS. 6, A3 and B3).

Furthermore, during size exclusion chromatography (SEC), the peptides PS1-70 and PS1-120 showed a retention volume of 12.16 ml and 10.92 ml, respectively (FIG. 6), indicative of an oligomer or a poorly compact protein. Light Scattering experiments carried out by SEC-MALS-QELS showed that, regardless of the retention volume, the peptides of the invention are present in solution as monodispersed monomeric proteins having molecular weights of 9.36±0.6 kDa for PS1-70 and 19.98±1.5 kDa for PS1-120, respectively, consistent with the theoretical ones (FIG. 8A, 8B).

The secondary structure of the peptides PS1-70 and PS1-120 was then analysed by circular dichroism (CD). The Far-UV CD spectrum obtained showed negative molar ellipticity values at 198 and 190 nm for both tested peptides. However, the ellipticity values observed at 200 and 222 nm are indicative of some secondary structure (FIG. 8C, 8D). The above characteristics are typical of a disordered protein having large unstructured portions. This is probably related both to the high number of acidic residues (negatively charged at physiological pH) responsible for the intrinsic repulsion, and to the low content of hydrophobic residues which generally assist proteins in folding correctly.

### Example 3: Induction of Defence Gene Expression in Plants by the Peptides According to the Invention

In order to test the biological activity of the peptides of the invention, the present inventors carried out studies to measure the expression of defence genes in tomato plants (Solanum lycopersicum) after application of the peptides PS1-70 and PS1-120 on these plants. Said peptides were assayed at picomolar (pM) and femtomolar (fM) concentrations in 1×PBS buffer (0.14 M NaCl, 0.0027 M KCl, 0.01 M phosphate buffer, pH 7.4) by applying 2 μl of the aqueous composition comprising the peptides on several points on the upper side of expanded leaves of four-week-old tomato plants.

Leaf samples were taken 6 hours and 24 hours after application of the peptides of the invention to undergo RNA extraction and subsequent gene expression analysis. In particular, four genes known to be related to plant defence were selected and tested: two early-expression genes active in the octadecanoid biosynthesis pathway leading to the formation of Jasmonic Acid (JA), such as the Lipoxygenase C gene (Lox C) and the allene oxide synthase gene (AOS), and two late-expression genes such as the proteinase I inhibitor (Pin I) and proteinase II inhibitor (Pin II) genes.

All assayed genes were significantly over-expressed following exogenous application of both peptides of the invention at both concentrations tested (FIG. 9).

### Example 4: The Peptides According to the Invention Promote Resistance to Biotic Stress in Plants

In order to demonstrate that the peptides of the invention are capable of promoting the resistance of plants against pathogenic organisms, the present inventors carried out studies for assessing the effects on herbivorous insects or phytopathogenic fungi resulting from the treatment of plants with peptides having the amino acid sequences SEQ ID NO.1 (PS1-70), 2 (PS1-120), 3, 5 and 8. More specifically, the present inventors monitored two different parameters, namely changes in weight gain and survival rate of larvae of Spodoptera littoralis, a lepidopteran that produces considerable damage to tomato plants, and the colonization of the plants by the phytopathogenic necrotrophic Botrytis cinerea fungus, an agent that causes tomato grey mould, and the parasitic Alternaria alternata fungus.

Experiments on Spodoptera littoralis Larvae

Briefly, S. littoralis larvae were grown in a climate chamber at 25° C., 70% relative humidity (RH), with a 16-hour light and 8-hour dark photoperiod and fed with an artificial diet until completion of the first moult. For the bioassays, 150 larvae for each thesis were grown on tomato leaves for the entire duration of the second instar to adapt them to the different diet. The newly moulted third-instar larvae up to the moult stage were fed with plants on which a composition comprising peptides PS1-70 and PS1-120 had been applied at 100 pM and 100 fM concentrations in 1×PBS buffer. Larvae fed with plants treated with 1×PBS buffer only were used as a control. Bioassays were carried out under the same environmental conditions, in 32-well plastic trays containing 1.5% (w/v) agar and 0.005% (w/v) methyl parahydroxybenzoate, which are useful for creating a humid environment for maintaining cell turgor of tomato leaves. Each experimental group consisted of 32 larvae. The survival of the larvae was monitored daily, and their weight every other day. Larvae fed with leaves treated with the peptides of the invention showed weight loss throughout the bioassay period compared to those fed with the control leaves, with a significant difference for both concentrations starting as early as the third day. In particular, on day 15 of the bioassay, for the 100 pM concentration, larvae fed with the control leaves had an average weight of 38 mg, whereas those fed with leaves treated with the peptides PS1-70 and PS1-120 had an average weight of 15 and 20 mg, respectively (FIG. 10A). In the bioassays carried out using 100 fM concentration of the peptides of the invention, on day 13 the larvae fed with the control leaves had an average weight of 44 mg, whereas those fed with leaves treated with the peptides PS1-70 and PS1-120 had an average weight of 11 and 12 mg, respectively (FIG. 10C). A significant decrease in the survival of larvae fed with leaves of plants treated with the peptides of the invention compared to those fed with leaves of the control plants was also observed for both assays (FIGS. 10B and 10D). In fact, on day 15 of the bioassay a survival rate of 96.87% was observed for larvae fed with the control leaves, and survival rates of 34.37% and 31.25%, respectively, were observed for those fed with leaves treated with the peptides of the invention at 100 pM concentrations (FIG. 10B). In the bioassay carried out at the 100 fM concentration, on day 13, a 100% survival rate was observed for larvae fed with the control leaves, and 0% and 21, 87% survival rates, respectively, were observed for those fed with leaves treated with the peptides of the invention (FIG. 10D).

The same assay scheme was carried out by the present inventors by feeding S. littoralis larvae with leaves treated with the peptide PS1-70 and with peptides having the sequences SEQ ID NO. 3, 5, and 8 at femtomolar concentration. Larvae fed with the treated leaves showed a significant reduction in their weight from day 3 compared to the control leaves (FIG. 11). In particular, on day 13 of the bioassay, larvae fed with the control leaves had an average weight of 59 mg, instead those treated with the peptides PS1-70, SEQ ID NO. 8, 3, and 5 had an average weight of 22 mg, 13 mg, 24 mg and 21 mg, respectively (FIG. 11A). A significant decrease was also observed in the survival of larvae fed with peptide-treated leaves compared to those fed with the control leaves (FIG. 11B). In fact, on day 13 of the bioassay a survival rate of 100% was observed for larvae fed with the control leaves, and survival rates of 31.25%, 15.62%, 43.75% and 15.62%, respectively, were observed for those fed with leaves treated with the peptides PS1-70, SEQ ID NO. 8, SEQ ID NO. 3, and SEQ ID NO. 5 (FIG. 11B).

Experiments on Botrytis cinerea and Alternaria alternata

For the performance of assays on the phytopathogenic Botrytis cinerea and Alternaria alternata fungi, the present inventors used spores of this microorganism obtained from cultures on solid PDA (Potato Dextrose Agar) sporulation substrate. The plates were inoculated with 20 μl of a conidial suspension at a concentration of 1×10⁶ spores/ml and incubated for 15 days at 25° C. in the presence of diffused light, so as to obtain complete sporulation. The spores were then collected in 5 ml of sterile water and, in order to remove the mycelium, were filtered through glass wool, washed with sterile distilled water and collected by centrifugation at room temperature. The concentration of spores suitable for inoculation (10⁵-10⁷ spores/ml) was determined by the serial dilution method using a Burker cell counting chamber for spore counting.

The assay was carried out on a detached leaf by taking a compound leaf for each treated tomato plant and for each control plant. Each leaflet of the compound leaf was marked with 3 markers in order to guide the subsequent application of the spores and detection of the pathogen developing necrotic areas.

The leaves were treated by applying 2 μl of a composition comprising the peptides PS1-70 and PS1-120 of the invention at a concentration of 100 pM, 100 fM or 1×PBS for the control leaves. After 6 hours, the time necessary for the perception of the peptides, the leaves were detached from the plant and inoculations with 10 μl of spore solution were carried out in the internerve spaces and near the previously marked points. Monitoring was carried out by measuring the necrosis areas (expressed in mm²) 1, 3, 5, 8 days after inoculation of the pathogen. The necrosis areas recorded on the control leaves were much larger than those detected on plants treated with the peptides of the invention. This difference increased as a function of the time elapsed since inoculation. The resistance inducing effect of the picomolar treatment is already statistically significant from the first day of inoculation of the pathogen up to the eighth day where values of 33 mm² were reached on the control leaves, unlike the treated leaves where values not exceeding 18 mm² were recorded for both peptides (FIG. 12A).

Similar results were observed following application of femtomolar concentrations of the peptides object of the invention. In particular, a strong reduction in the development of necrotic areas on the treated leaves compared to those on the control leaves was observed as early as day 1 of inoculation of the phytopathogenic fungus. This reduction was maintained until day 8 where values of 20 mm² were reached on the control leaves, and values of 6.69 mm² and 5.19 mm² were reached on leaves treated with PS1-70 and PS1-120, respectively (FIG. 12B).

The present inventors also carried out tests aimed at determining the effects of exogenous application of the peptides of the invention, at picomolar and femtomolar concentrations, against the development of the necrotrophic fungus on Solanum melongena (eggplant) plants (FIG. 12C) and Vitis vinifera (vine) plants (FIG. 12D).

As shown in FIG. 12C, the tests performed showed a significant reduction in fungal colonization of the treated plants compared to the control plants, and the most marked effect was observed following treatment with the lowest concentration of the peptides of the invention. The positive effect of the treatment was statistically significant from day 3 of inoculation of the pathogen to day 8 where values of 11 mm² were reached on the control leaves, unlike the treated leaves where values of 8.4 and 6.0 mm² were recorded on leaves treated with the peptides PS1-70 and PS1-120 at a concentration of 100 pM, and values of 6.2 and 4.10 mm² were recorded on leaves treated with said peptides at a concentration of 100 fM (FIG. 12C). Similar results were obtained on vine plants (FIG. 12D). The positive effect of the treatment was already statistically significant from day 1 of inoculation of the pathogen to day 8 where values of 43 mm² were reached on the control leaves, and values of 16.7 and 13.6 mm², respectively, were reached on leaves treated with the peptides PS1-70 and PS1-120 at a concentration of 100 pM, and values of 12 and 11.8 mm² were reached on leaves treated with said peptides at a concentration of 100 fM (FIG. 12D). Similar results were obtained on olive trees.

The effect of application of femtomolar concentrations of the peptides having the sequences SEQ ID NO. 3, 5 and 8 on tomato leaves was also assessed against the development of the B. cinerea fungus.

As shown in FIG. 13, the necrosis areas recorded on the control leaves were much larger than those detected on plants treated with the peptides of the invention. The positive effect of the treatment was already statistically significant from day 1 of inoculation of the pathogen and continued until day 8 where values of 11.72 mm² were reached on the control leaves, and values of 7.69 mm², 7.67 mm², 8.89 mm² and 6.81 mm² were reached on leaves treated with the peptides PS1-70, SEQ ID NO.8, SEQ ID NO.3, SEQ ID NO.5, respectively, at a concentration of 100 fM (FIG. 13).

The present inventors also performed assays aimed at determining the effects of exogenous application of the peptides of the invention on tomato plants against the development of the necrotrophic Alternaria alternata fungus (FIG. 14).

As shown in FIGS. 14A and 14B, the assays performed showed a significant reduction in fungal colonization of the treated plants compared to the control plants. The positive effect of the treatment was statistically significant from day 1 of inoculation of the pathogen to day 8 where values of 24 mm² were reached on the control leaves, unlike the treated leaves where values of 8.4 and 8.0 mm² were recorded on leaves treated with the peptides PS1-70 and PS1-120 at a concentration of 100 fM (FIG. 14A); and values of 17 mm² were recorded on the control leaves and values of 8.3 mm², 7.9 mm², 8.9 mm², and 7.8 mm² were recorded on leaves treated with the peptides PS1-70, SEQ ID NO.8, SEQ ID NO.3 and SEQ ID NO.5, respectively, at a concentration of 100 fM (FIG. 14B).

Activity of the Peptides According to the Invention in Combination with Trichoderma T22 Spores

Further studies were carried out by the present inventors in order to determine the effects on tomato plants of treatment with the peptides of the invention, at femtomolar concentrations, in combination with Trichoderma T22 spores in reducing the survival rate of S. littoralis larvae (A1, A2, A3) and the development of the necrotrophic B. cinerea (B1) and A. alternata (B2) fungi (FIG. 15).

Tomato seeds were treated with a suspension of Trichoderma harzianum T22 strain spores (1×10⁷ spores/ml) or with water for the controls, allowed to dry and germinated in the dark on sterile adsorbent paper at 24° C. 4-week-old plant leaves were treated by applying 2 μl of a composition comprising the peptides Systemin (Sys), PS1-70 or PS1-120 at a concentration of 100 fM or 1×PBS on the control leaves. After 6 hours, the time necessary for the perception of the peptides, the leaves were detached from the plant to carry out both the assay with the S. littoralis larvae and the assay with the two necrotrophic fungi B. cinerea and A. alternata.

For the assay with the S. littoralis larvae, the survival of the larvae was monitored daily. To monitor the development of the two phytopathogenic fungi, on the other hand, measurements of the necrosis areas (expressed in mm²) were made 1, 3, 5, 8 days after the inoculation of 10 μl of spore solution (1×10⁶ spores/ml) on the previously treated tomato leaves.

FIG. 15 shows that treatment with the tested peptides in combination with Trichoderma T22 produces a surprising synergistic effect in counteracting the development and survival of the larvae and a significantly higher protection against the colonization by the two phytopathogenic fungi compared to treatments with Trichoderma T22 alone or with the peptides alone. The evidence of these effects increases as a function of time. Moreover, the effects produced by the peptides of the invention, used individually or in combination with the Trichoderma T22 spores, have been shown to be superior compared to the peptide systemin.

Seed Protective Effect of the Peptides According to the Invention

The present inventors also assessed the protective effect conferred by the treatment with the peptides object of the invention directly on the seed. Tomato seeds were treated with a composition comprising the peptides at a concentration of 100 fM or with 1×PBS for the controls, allowed to dry and germinated in the dark on sterile adsorbent paper at 24° C. 4-week-old plant leaves were detached from the plant and inoculated with 10 μl of spore solution in the internerve spaces and near the previously marked points. As shown in FIG. 16, the assay showed a significant reduction in fungal colonization on the leaves of plants born from seeds treated with PS1-70 and PS1-120 compared to control plants.

The positive effect of the treatment was statistically significant from day 1 of inoculation of the pathogen to day 8 where values of 11 mm² were reached on the control leaves, and values of 7.1 and 7.4 mm² were reached on leaves from plants born from seeds treated with the peptides PS1-70 and PS1-120 at a concentration of 100 fM (FIG. 16). Further studies were carried out by the present inventors in order to investigate the presence of any direct toxic effect of the peptides PS1-70 and PS1-120 on the tested pathogenic organisms. As can be seen from the data reported in the table in FIG. 21, the administration of the peptides of the invention orally or by injection in the epidermis of S. littoralis larvae had no effect on their survival and development.

Furthermore, as shown in FIGS. 22A and 22B, the growth of the B. cinerea fungus was not disrupted when the growth medium was added with the peptides PS1-70 and PS1-120 of the invention. Further studies were carried out on Trichoderma harzianum T22 strain and again, as shown in FIG. 22C, the addition of the peptides PS1-70 and PS1-120, at both the 100 pM and 100 fM concentrations, had no effect on the growth of the fungus. Instead, an increase in the growth of the T22 fungus was observed when the peptide PS1-70 was added to the growth medium, suggesting the possibility that the fungus uses this protein as a source of amino acids.

In summary, the experimental results described above show that the peptides object of the present invention are biologically active, and that exogenous application thereof promotes resistance to noxious insects and fungi in plants.

### Example 5: The Peptides According to the Invention Promote Resistance to Abiotic Stress in Plants

During their studies, the present inventors have also set up experiments aimed at verifying the efficacy of the peptides of the invention in promoting the resistance of plants against various abiotic stresses.

Through the experiments carried out, the inventors first found that the application of the peptides of the invention on tomato plants causes an increase in their biomass and favours the production of larger berries with a greater number of seeds. The results of subsequent experiments, as shown in FIG. 17, showed that the application of the peptide PS1-70 protects the tomato plant from salt stress. Briefly, 48 hours after irrigation with the PS1-70 peptide at a concentration of 100 pM, the treated plants, unlike the control plants, were able to activate a series of genes and transcription factors responsive to salt stress, thereby demonstrating that the peptide of the invention is capable of stimulating an alert state in the plant, known as priming. Moreover, in the presence of moderate salt stress (80 mM NaCl), tomato plants treated with the peptide of the invention were more tolerant to salt than the untreated control plants, showing in particular a greater induction of genes responsive to this stress (FIG. 17B). An experiment was also set up to verify the efficiency of the peptides PS1-70, PS1-120 and SEQ ID NO.5, when administered at femtomolar concentrations, in promoting tolerance to a high salt stress condition. As shown in FIG. 18, 8 days after irrigation with the peptides PS1-70, PS1-120 and SEQ ID NO.5, the treated plants, unlike the control plants, were able to activate a series of salt stress-responsive genes, thus confirming that the peptides of the invention are capable of stimulating the priming defence state even at femtomolar concentrations (FIGS. 18A1, 18B1 and 18C1). Moreover, tomato plants treated with the above peptides and 24 hours later irrigated for 7 days at a high salt stress level (150 mM NaCl) were more salt-tolerant than the untreated control plants, showing in particular a greater induction of the same genes responsive to this stress (FIGS. 18A2, 18B2 and 18C2).

The average proline content was also assessed in these plants. Proline is an osmoprotector produced in the free form in the plant cell in response to salt stress and water deficiency. In plants subjected to salt stress, this amino acid, in fact, participates in the regulation of the osmotic potential, in the protection of membranes from free radicals, in the regulation of the cytoplasmic pH, and in the protection of enzymes from denaturation.

As shown in FIG. 19, plants treated with the peptides of the invention in the presence of high salt stress (150 mM NaCl) exhibit a lower average proline content than the salinized control plants, thus demonstrating their lower perception of the osmotic stress caused by the salt.

A few biometric parameters are shown in FIG. 20, measured after 14 days of continuous salt stress (150 mM) and 15 days after irrigation with the peptides PS1-70, PS1-120 and SEQ ID NO.5 at 100 fM concentrations, and the respective controls. FIG. 20A shows that peptide-treated plants exhibited greater tolerance to stress than untreated control plants, in particular with a greater root surface (FIG. 20A). Furthermore, in the absence of salt, the plants treated with the peptides object of the invention, unlike the control plants, are able to promote the growth of the aerial part of the plant, thus confirming that the peptides of the invention have a biostimulant effect even at femtomolar concentrations (FIG. 20B).

What is claimed is:

1. An isolated peptide consisting of an amino acid sequence selected from the group consisting of SEQ ID NOs. 1, 2, 3-9, 23-26 and having biostimulant and bioprotective activity against abiotic and biotic stress in plants, the isolated peptide being optionally conjugated with a histidine tail at the amino-terminal end or at the carboxy-terminal end.

2. (canceled)

3. The isolated peptide of claim 1, wherein the amino-terminal end is modified by acetylation and/or the carboxy-terminal end is modified by amidation.

4. An isolated nucleic acid sequence encoding the isolated peptide of claim 1.

5. An expression vector comprising the nucleic acid sequence of claim 4.

6. A host cell comprising the expression vector of claim 5.

7. A method for preparing an isolated peptide consisting of an amino acid sequence selected from the group consisting of SEQ ID NOs. 1, 2, 3-9, 23-26 and having biostimulant and bioprotective activity against abiotic and biotic stress in plants, the isolated peptide being optionally conjugated with a histidine tail at the amino-terminal end or at the carboxy-terminal end, the method comprising culturing a host cell according to claim 6 under suitable conditions and for a time sufficient for the expression of the peptide and, optionally, recovering the peptide from the culture.

8. A biostimulant and bioprotective composition against abiotic and biotic stress in plants, comprising at least one peptide according to claim 1, and at least one adjuvant, stabilizer and/or preservative.

9. The biostimulant and bioprotective composition of claim 8, wherein the at least one peptide is present in a concentration ranging from 0.01 picomolar (pM) to 100 pM.

10. The biostimulant and bioprotective composition of claim 8, further comprising a microorganism selected from the group consisting of mycorrhizal fungi, saprophytic fungi, plant growth promoting bacteria, Bacillus thuringiensis spores, and any combination thereof.

11. The biostimulant and bioprotective composition according to of claim 8, which wherein the biostimulant and bioprotective composition is a water-based liquid composition.

12. The biostimulant and bioprotective composition of claim 8, wherein the biostimulant and bioprotective composition is in lyophilized form.

13. A method for increasing resistance to biotic and/or abiotic stress in a plant, comprising applying a biostimulant and bioprotective composition according to claim 8 to the plant, parts of the plant, plant propagation material, and/or plant growth site.

14. The method of claim 13, wherein the biostimulant and bioprotective composition is applied by spraying, irrigation or is supplied in a hydroponic solution.

15. The method of claim 13, wherein the plant is a plant belonging to the Solanaceae, Vitaceae, Rosaceae or Oleaceae family.

16. A method for increasing resistance to abiotic and/or biotic stress in a plant, the method comprising applying to the plant the isolated peptide of claim 1.


SEQUENCE LISTING (26 sequences)

SEQ ID NO: 1 - protein, 70 aa, Artificial Sequence
  Peptide PS1-70
MGTPSYDIKNKGDDMQEEPKVKLHHEKGGDEKEKIIEKETPSQDINNKDTISSYVLRDDTQEIPKMEHEE

SEQ ID NO: 2 - protein, 120 aa, Artificial Sequence
  Peptide PS1-120
MGTPSYDIKNKGDDMQEEPKVKLHHEKGGDEKEKIIEKETPSQDINNKDTISSYVLRDDTQEIPKMEHEEGGYVKEKIVEKETISQYIIKIEGDDDAQEKLKVEYEEEEYEKEKIVEKET

SEQ ID NO: 3 - protein, 9 aa, Artificial Sequence
  Peptide
EKETPSQDI

SEQ ID NO: 4 - protein, 9 aa, Artificial Sequence
  Peptide
EKETISQYI

SEQ ID NO: 5 - protein, 16 aa, Artificial Sequence
  Peptide
DDMQEEPKVKLHHEKG

SEQ ID NO: 6 - protein, 14 aa, Artificial Sequence
  Peptide
DDTQEIPKMEHEEG

SEQ ID NO: 7 - protein, 14 aa, Artificial Sequence
  Peptide
DDAQEKLKVEYEEE

SEQ ID NO: 8 - protein, 33 aa, Artificial Sequence
  Peptide
DDMQEEPKVKLHHEKGGDEKEKIIEKETPSQDI

SEQ ID NO: 9 - protein, 31 aa, Artificial Sequence
  Peptide
DDTQEIPKMEHEEGGYVKEKIVEKETISQYI

SEQ ID NO: 10 - DNA, 210 bp, Artificial Sequence
  Sequence encoding PS1-70
atgggaactccttcatatgatatcaaaaacaaaggagatgacatgcaagaagaaccaaaggtgaaacttcaccatgagaagggaggagatgaaaaggaaaaaataattgaaaaagagactccatcccaagatatcaacaacaaagataccatctcttcatatgttttaagagatgatacacaagaaataccaaagatggaacatgaggag

SEQ ID NO: 11 - DNA, 360 bp, Artificial Sequence
  Sequence encoding PS1-120
atgggaactccttcatatgatatcaaaaacaaaggagatgacatgcaagaagaaccaaaggtgaaacttcaccatgagaagggaggagatgaaaaggaaaaaataattgaaaaagagactccatcccaagatatcaacaacaaagataccatctcttcatatgttttaagagatgatacacaagaaataccaaagatggaacatgaggagggaggatatgtaaaggagaaaattgttgaaaaggagactatatcccaatatatcatcaagattgaaggagatgatgatgcacaagaaaaactaaaggttgagtatgaggaggaagaatatgaaaaagagaaaatagttgaaaaagagact

SEQ ID NO: 12 - DNA, 436 bp, Artificial Sequence
  PS1-70 recombinant insert
gagatctcgatcccgcgaaattaatacgactcactataggggaattgtgagcggataacaattcccctctagaaataattttgtttaactttaagaaggagatataccatgaaacatcaccatcaccatcaccccatgagcgattacgacatccccactactgagaatctttattttcagggcgccatgggaactccttcatatgatatcaaaaacaaaggagatgacatgcaagaagaaccaaaggtgaaacttcaccatgagaagggaggagatgaaaaggaaaaaataattgaaaaagagactccatcccaagatatcaacaacaaagataccatctcttcatatgttttaagagatgatacacaagaaataccaaagatggaacatgaggagtagtaactcgagcaccaccaccaccaccactgagatccgg

SEQ ID NO: 13 - DNA, 513 bp, Artificial Sequence
  PS1-120 recombinant insert
ataattttgtttaactttaagaaggagatataccatgaaacatcaccatcaccatcaccccatgagcgattacgacatccccactactgagaatctttattttcagggcgccatgggaactccttcatatgatatcaaaaacaaaggagatgacatgcaagaagaaccaaaggtgaaacttcaccatgagaagggaggagatgaaaaggaaaaaataattgaaaaagagactccatcccaagatatcaacaacaaagataccatctcttcatatgttttaagagatgatacacaagaaataccaaagatggaacatgaggagggaggatatgtaaaggagaaaattgttgaaaaggagactatatcccaatatatcatcaagattgaaggagatgatgatgcacaagaaaaactaaaggttgagtatgaggaggaagaatatgaaaaagagaaaatagttgaaaaagagacttagtaactcgagcaccaccaccaccaccactgagatccggc

SEQ ID NO: 14 - DNA, 18 bp, Artificial Sequence
  Histidine tag coding sequence
catcaccatcaccatcac

SEQ ID NO: 15 - protein, 26 aa, Artificial Sequence
  Histidine tag
MKHHHHHHPMSDYDIPTTENLYFQGA

SEQ ID NO: 16 - DNA, 21 bp, Artificial Sequence
  TEV site
gagaatctttattttcagggc

SEQ ID NO: 17 - DNA, 33 bp, Artificial Sequence
  primer P11F1 forward
cgcgcgccatgggaactccttcatatgatatca

SEQ ID NO: 18 - DNA, 41 bp, Artificial Sequence
  primer P11R1 reverse
cgcgcgctcgagttactactcctcatgttccatctttggta

SEQ ID NO: 19 - DNA, 48 bp, Artificial Sequence
  primer reverse P11R3
cgcgcgctcgagttactaagtctctttttcaactattttctctttttc

SEQ ID NO: 20 - DNA, 30 bp, Artificial Sequence
  Sequence beetween histidine tag and TEV site
cccatgagcgattacgacatccccactact

SEQ ID NO: 21 - protein, 96 aa, Artificial Sequence
  Peptide PS1-70 with histidine tag
MKHHHHHHPMSDYDIPTTENLYFQGAMGTPSYDIKNKGDDMQEEPKVKLHHEKGGDEKEKIIEKETPSQDINNKDTISSYVLRDDTQEIPKMEHEE

SEQ ID NO: 22 - protein, 146 aa, Artificial Sequence
  Peptide PS1-120 with histidine tag
MKHHHHHHPMSDYDIPTTENLYFQGAMGTPSYDIKNKGDDMQEEPKVKLHHEKGGDEKEKIIEKETPSQDINNKDTISSYVLRDDTQEIPKMEHEEGGYVKEKIVEKETISQYIIKIEGDDDAQEKLKVEYEEEEYEKEKIVEKET

SEQ ID NO: 23 - protein, 14 aa, Artificial Sequence
  Peptide
DDAQEKPKVEHEEG

SEQ ID NO: 24 - protein, 9 aa, Artificial Sequence
  Peptide
DKETPSQDI

SEQ ID NO: 25 - protein, 31 aa, Artificial Sequence
  Peptide
DDAQEKLKVEYEEEEYEKEKIVEKETPSQDI

SEQ ID NO: 26 - protein, 24 aa, Artificial Sequence
  Peptide
DDAQEKPKVEHEEGDDKETPSQDI